(19) United States
(12) Patent Application Publication
(10) Pub. No.: US 2025/0000607 A1
(43) Pub. Date: Jan. 2, 2025

(54) VACUUM ASSISTED BIOPSY DEVICE WITH VALVE-CONTROLLED VENTING AND INTEGRATED MARKER DELIVERY

(71) Applicant: Bard Peripheral Vascular, Inc., Franklin Lakes, NJ (US)

(72) Inventors: Laura Cavanagh, Limerick (IE); Jamie Collins, Mungret (IE); Koltin Glaspie, Chandler, AZ (US); Dwayne Griffin, Limerick (IE); James Howard, Castletroy (IE); Robert Meehan, Dublin (IE); Andrew Sharpe, Clarina (IE); Marvin Clyde C. Vergara, Avondale, AZ (US)

(73) Assignee: Bard Peripheral Vascular, Inc., Franklin Lakes, NJ (US)

(21) Appl. No.: 18/711,940

(22) PCT Filed: Nov. 23, 2022
(86) PCT No.: PCT/US2022/080439

§ 371 Date: May 21, 2024

Related U.S. Application Data
(60) Provisional application No. 63/283,159, filed on Nov. 24, 2021.

(51) Int. Cl. A61B 90/00; A61B 10/02

(52) U.S. Cl. CPC A61B 90/39; A61B 10/0266; A61B 2010/0208; A61B 2090/3908; A61B 2090/3987

(57) ABSTRACT

A marker delivery assembly for a biopsy device includes an integration module and a marker delivery module. The integration module includes a connector defining a cavity and a neck that extends outwardly from the cavity in a longitudinal direction. A transmission cover having a shaft is disposed about the neck, and further includes a spindle gear and a drum gear. A bevel drum is rotatably coupled to the drum gear and a pushrod is engaged to the bevel drum. The marker delivery module includes a marker housing releasably coupled to the integration module. A marker delivery cartridge engages the marker housing, and is rotatable between an initial position and a delivery position. The pushrod extends through the marker delivery cartridge and forces at least one marker into the biopsy device.

### CROSS-REFERENCE TO RELATED APPLICATIONS

The present application claims the priority benefit of U.S. Provisional Application No. 63/283,159, entitled “Vacuum Assisted Biopsy Device with Valve-Controlled Venting and Integrated Marker Delivery” and filed Nov. 24, 2021, the entire contents of which is incorporated by reference herein.

### TECHNICAL FIELD

The present disclosure relates to biopsy devices, and, more particularly, to a single insertion multiple sample biopsy device.

### BACKGROUND

A biopsy may be performed on a subject to help in determining whether the tissue in a region of interest includes cancerous cells, for example. One biopsy technique used to evaluate breast tissue, for example, involves inserting a biopsy probe into the breast tissue region of interest to capture one or more tissue samples from the region. Such a biopsy technique often utilizes a vacuum to pull the tissue to be sampled into a sample notch of the biopsy probe, after which the tissue is severed and collected. Efforts continue in the art to improve the ability of the biopsy device to sever a tissue sample, and to transport the severed tissue sample to a sample collection container.

Many biopsy techniques may further involve deploying a marker that identifies the region from which tissue has been severed and collected, such that the region may be easily identified and monitored. However, difficulties often arise during placement of the marker, particularly when biopsy techniques are employed that utilize separate biopsy and marker delivery devices.

Accordingly, a need exists for a biopsy device that has the ability to promote effective severing of a tissue sample and/or effective transport of the tissue sample to a sample collection container and easily deploy a marker to a region from which tissue has been severed.

### SUMMARY

An object of the present disclosure is to provide a biopsy device that can effectively sever a tissue sample and easily deploy a marker to a region from which the tissue has been severed.

In one embodiment, a marker delivery assembly is disclosed. The marker delivery assembly includes an integration module having a connector defining a cavity and a neck that extends outwardly from the cavity in a longitudinal direction. A transmission cover having a shaft is coaxially disposed about the neck of the connector, the shaft further extending between a distal end having a spindle gear and a proximal end having a drum gear. A bevel drum is rotatably coupled to the drum gear, and a pushrod is frictionally engaged to the bevel drum. The marker delivery assembly further includes a marker delivery module having a marker delivery housing that fits within the cavity of the connector to releasably couple the marker delivery module to the integration module. The marker delivery housing includes at least one coupling rod and at least one fulcrum rod. The marker delivery module further includes a marker delivery cartridge that is rotatable between an initial position in which the marker delivery cartridge engages the at least one coupling rod, and a delivery position in which the marker delivery cartridge is rotated about the at least one fulcrum rod. The marker delivery cartridge further includes a chamber that houses a plurality of markers. When the marker delivery cartridge is in the delivery position, rotation of the bevel drum causes the pushrod to unwind, such that the pushrod extends through the chamber of the marker delivery cartridge and forces at least one of the plurality of markers into the biopsy device.

In another embodiment, a biopsy device is disclosed. The biopsy device includes a driver assembly and a biopsy probe assembly releasably attached to the driver assembly. The biopsy probe assembly includes a cutter cannula and a sample notch cannula coaxially arranged along a longitudinal axis. The cutter cannula is positioned inside the sample notch cannula to define a fluid pathway between the cutter cannula and the sample notch cannula. The biopsy device further includes a marker delivery assembly includes an integration module having a connector defining a cavity and a neck that extends outwardly from the cavity in a longitudinal direction. A transmission cover having a shaft is coaxially disposed about the neck of the connector, the shaft further extending between a distal end having a spindle gear and a proximal end having a drum gear. A bevel drum is rotatably coupled to the drum gear, and a pushrod is frictionally engaged to the bevel drum. The marker delivery assembly further includes a marker delivery module having a marker delivery housing that fits within the cavity of the connector to releasably couple the marker delivery module to the integration module. The marker delivery housing includes at least one coupling rod and at least one fulcrum rod. The marker delivery module further includes a marker delivery cartridge that is rotatable between an initial position in which the marker delivery cartridge engages the at least one coupling rod, and a delivery position in which the marker delivery cartridge is rotated about the at least one fulcrum rod. The marker delivery cartridge further includes a chamber that houses a plurality of markers. The biopsy device further includes a sample basket assembly releasably attached to the marker delivery module of the marker delivery assembly, the sample basket assembly having a vacuum port for connecting the fluid pathway in fluid communication with a vacuum source, a venting line for connecting the fluid pathway to atmosphere, and a saline port for connecting the fluid pathway to a saline source.

In yet another embodiment, a method of using a biopsy device is disclosed. The method involves releasably coupling a marker delivery assembly to the biopsy device, where the marker delivery assembly including a bevel drum coupled to a pushrod, and a marker delivery cartridge rotatable between an initial position and a delivery position. The method further involves rotating the marker delivery cartridge from the initial position to the delivery position. Once in the delivery position, the method may involve rotating the bevel drum, such that the rotation of the bevel drum extends the pushrod into and through the marker delivery cartridge. As the pushrod extends, the method may involve contacting, with the pushrod, at least one of a plurality of markers housed within the marker delivery cartridge. Once the pushrod connects the at least one of the plurality markers, the method may involve pushing, with the pushrod, the at least one of the plurality of markers housed within the marker delivery cartridge out of the marker delivery cartridge and into the biopsy device. Finally, the method may involve deploying, using the pushrod, the at least one of the plurality of markers from the biopsy device.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of a biopsy device having a biopsy probe assembly attached to a biopsy driver assembly and a sample basket assembly, according to one or more embodiments of the present disclosure;

FIG. 2 schematically depicts a front side view of the biopsy device of FIG. 1, according to one or more embodiments of the present disclosure;

FIG. 3 schematically depicts a perspective view of the biopsy device of FIG. 1, according to one or more embodiments of the present disclosure;

FIG. 4A schematically depicts a bottom side view of the biopsy driver assembly of FIG. 1, according to one or more embodiments of the present disclosure;

FIG. 4B schematically depicts a prime/fire operation of a piercing module of the biopsy driver assembly of FIG. 4A in a disengaged position, according to one or more embodiments of the present disclosure

FIG. 4C schematically depicts a prime/fire operation of a piercing module of the biopsy driver assembly of FIG. 4A in a primed position, according to one or more embodiments of the present disclosure

FIG. 4D schematically depicts a prime/fire operation of a piercing module of the biopsy driver assembly of FIG. 4A in a firing position, according to one or more embodiments of the present disclosure;

FIG. 5 schematically depicts a longitudinal cross-sectional view of the biopsy device of FIG. 1 along line/plane 5-5 with the biopsy probe assembly attached to the biopsy driver assembly, according to one or more embodiments of the present disclosure;

FIG. 6A schematically depicts a portion of the biopsy device of FIG. 1 with the biopsy probe assembly separated from the biopsy driver assembly, according to one or more embodiments of the present disclosure;

FIG. 6B schematically depicts a close up view of retaining hooks used to attach the biopsy probe assembly and biopsy driver assembly of FIG. 1, according to one or more embodiments of the present disclosure;

FIG. 7A schematically depicts a top side cross-sectional view of the biopsy probe assembly of FIG. 1, according to one or more embodiments of the present disclosure;

FIG. 7B schematically depicts an enlargement of a portion of the top side cross-sectional view of the biopsy probe assembly of FIG. 7A, according to one or more embodiments of the present disclosure;

FIG. 8A schematically depicts an exploded view of the sample basket assembly of FIG. 1, according to one or more embodiments of the present disclosure;

FIG. 8B schematically depicts a perspective view of a probe interface of the sample basket assembly of FIG. 8A, according to one or more embodiments of the present disclosure;

FIG. 8C schematically depicts an outer basket housing of the sample basket assembly of FIG. 8A, according to one or more embodiments of the present disclosure

FIG. 8D schematically depicts a rear-side view of the outer basket housing of the sample basket assembly of FIG. 8A, according to one or more embodiments of the present disclosure

FIG. 8E schematically depicts a perspective side view the outer basket housing of the sample basket assembly of FIG. 8A, according to one or more embodiments of the present disclosure;

FIG. 8F schematically depicts a hinge basket assembly of the sample basket assembly of FIG. 8A in a closed configuration, according to one or more embodiments of the present disclosure

FIG. 8G schematically depicts a hinge basket assembly of the sample basket assembly of FIG. 8A in an open configuration, respectively, according to one or more embodiments of the present disclosure;

FIG. 8H schematically depicts a perspective view of a rear housing of the sample basket assembly of FIG. 8A, according to one or more embodiments of the present disclosure;

FIG. 9A schematically depicts a connector of an integration module of a marker delivery assembly, according to one or more embodiments of the present disclosure;

FIG. 9B schematically depicts a bevel drum of an integration module of a marker delivery assembly, according to one or more embodiments of the present disclosure;

FIG. 9C schematically depicts an adapter of an integration module of a marker delivery assembly, according to one or more embodiments of the present disclosure;

FIG. 10 schematically depicts a perspective view of a marker delivery module of the marker delivery assembly, according to one or more embodiments of the present disclosure;

FIG. 11A schematically depicts the marker delivery module of FIG. 10 in an initial position, according to one or more embodiments of the present disclosure

FIG. 11B schematically depicts the marker delivery module of FIG. 10 in a delivery position, according to one or more embodiments of the present disclosure

FIG. 11C schematically depicts the marker delivery module of FIG. 10 delivering a marker to the integration module of FIGS. 9A-9C, according to one or more embodiments of the present disclosure;

FIG. 12A schematically depicts the marker delivery assembly coupled to the biopsy probe assembly of FIGS. 7A-7B in a disengaged position, according to one or more embodiments of the present disclosure

FIG. 12B schematically depicts the marker delivery assembly coupled to the biopsy probe assembly of FIGS. 7A-7B in an engaged position, according to one or more embodiments of the present disclosure

FIG. 12C schematically depicts a cross-section view of the marker delivery assembly delivering a marker to a target site, according to one or more embodiments of the present disclosure;

FIG. 13A schematically depicts a first step of an installation process for coupling the marker delivery assembly on the biopsy device of FIG. 1, according to one or more embodiments of the present disclosure;

FIG. 13B schematically depicts a second step of an installation process for coupling the marker delivery assembly on the biopsy device of FIG. 1, according to one or more embodiments of the present disclosure;

FIG. 13C schematically depicts a third step of an installation process for coupling the marker delivery assembly on the biopsy device of FIG. 1, according to one or more embodiments of the present disclosure;

FIG. 14 schematically depicts a perspective view of a biopsy device station, according to one or more embodiments of the present disclosure;

FIG. 15A schematically depicts a graphical user interface for initialization of the biopsy device station of FIG. 14, according to one or more embodiments of the present disclosure;

FIG. 15B schematically depicts a graphical user interface for initialization set up of the biopsy device station of FIG. 14, according to one or more embodiments of the present disclosure;

FIG. 15C schematically depicts a graphical user interface for installation of a cassette of the biopsy device station of FIG. 14, according to one or more embodiments of the present disclosure;

FIG. 15D schematically depicts a graphical user interface for set up of a vacuum canister of the biopsy device station of FIG. 14, according to one or more embodiments of the present disclosure;

FIG. 15E schematically depicts a graphical user interface for set up of a vacuum of the biopsy device station of FIG. 14, according to one or more embodiments of the present disclosure;

FIG. 15F schematically depicts a graphical user interface for set up of a foot switch of the biopsy device station of FIG. 14, according to one or more embodiments of the present disclosure;

FIG. 15G schematically depicts a graphical user interface for set up of the biopsy driver assembly of the biopsy device station of FIG. 14, according to one or more embodiments of the present disclosure;

FIG. 15H schematically depicts a graphical user interface for set up of the biopsy probe assembly of the biopsy device station of FIG. 14, according to one or more embodiments of the present disclosure;

FIG. 15I schematically depicts a graphical user interface for set up of a saline bag of the biopsy device station of FIG. 14, according to one or more embodiments of the present disclosure;

FIG. 15J schematically depicts a graphical user interface for completion of set up tasks of the biopsy device station of FIG. 9, according to one or more embodiments of the present disclosure;

FIG. 16 schematically depicts a graphical user interface of the station during operation of the biopsy station of FIG. 9, according to one or more embodiments of the present disclosure;

Corresponding reference characters indicate corresponding parts throughout the several views. The exemplifications set out herein illustrate at least one embodiment of the present disclosure, and such exemplifications are not to be construed as limiting the scope of the present disclosure in any manner.

### DESCRIPTION OF EMBODIMENTS

Embodiments disclosed herein relate to biopsy devices and marker delivery assemblies for biopsy devices. In particular, the marker delivery assembly may include an integration module having a connector defining a cavity and a neck that extends outwardly from the cavity in a longitudinal direction. A transmission cover having a shaft may be coaxially disposed about the neck of the connector, the shaft further extending between a distal end having a spindle gear and a proximal end having a drum gear. A bevel drum may be rotatably coupled to the drum gear, and a pushrod may be frictionally engaged to the bevel drum. The marker delivery assembly further includes a marker delivery module having a marker delivery housing that fits within the cavity of the connector to releasably couple the marker delivery module to the integration module. The marker delivery housing includes at least one coupling rod and at least one fulcrum rod. The marker delivery module further includes a marker delivery cartridge that is rotatable between an initial position in which the marker delivery cartridge engages the at least one coupling rod, and a delivery position in which the marker delivery cartridge is rotated about the at least one fulcrum rod. The marker delivery cartridge further includes a chamber that houses a plurality of markers. When the marker delivery cartridge is in the delivery position, rotation of the bevel drum causes the pushrod to unwind, such that the pushrod extends through the chamber of the marker delivery cartridge and forces at least one of the plurality of markers into the biopsy device. In these embodiments, a user may ensure that the at least one of the plurality of markers is correctly positioned at a severed tissue site, such that the severed tissue site may be easily identified in future biopsy procedures and monitored.

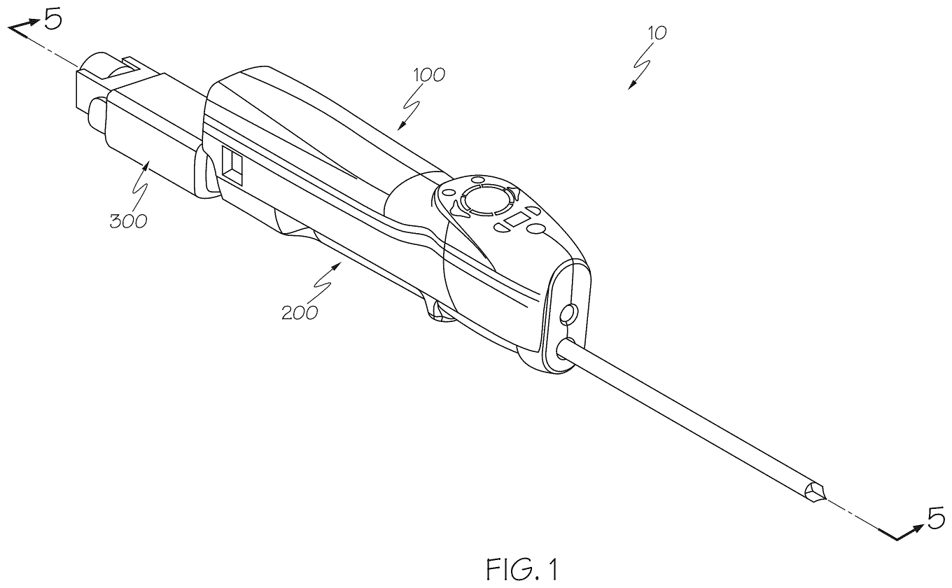

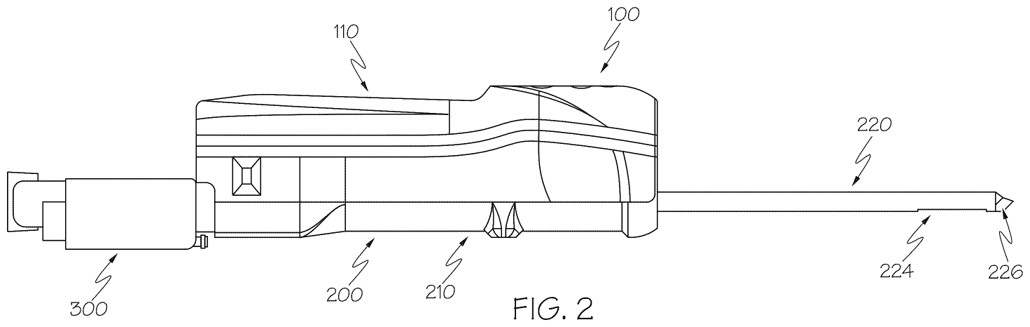

Referring now to the drawings, and more particularly to FIGS. 1 and 2, there is shown a vacuum assisted biopsy device 10 which may generally include a non-invasive, e.g., non-disposable, biopsy driver assembly 100 and an invasive, e.g., disposable, biopsy probe assembly 200. As used herein, the term “non-disposable” is used to refer to a device that is intended for use on multiple subjects during the lifetime of the device, and the term “disposable” is used to refer to a device that is intended to be disposed of after use on a single subject. The biopsy driver assembly 100 may be configured to be releasably attached to the biopsy probe assembly 200. As used herein, the term “releasably attached” means a configuration that facilitates an intended temporary connection followed by selective detachment involving a manipulation of disposable biopsy probe assembly 200 relative to biopsy driver assembly 100, without the need for tools. As further illustrated in FIGS. 1 and 2, the biopsy device 10 may also include a sample basket assembly 300, such as part of the disposable biopsy probe assembly 200.

Referring to FIGS. 2-4A, the biopsy driver assembly 100 may include a driver housing 110 that is configured and ergonomically designed to be grasped by a user, an electromechanical power source 120 and a controller 130. The controller 130 may include a plurality of control buttons providing user control over various functions of biopsy device 10. In embodiments the controller 130 may include visual/aural indicators. In embodiments, the controller 130 further includes any number of computer components, integrated circuit, chipsets, or the like for controlling various functionality described herein. In some embodiments, control elements are instead or in addition to those incorporated in the controller 130 part of a console (e.g., a computer) communicatively coupled to the controller 130 (such as via a wired or wireless connection). In some embodiments, the visual/aural indicators may provide visual/aural feedback of the status of one or more conditions and/or positions of components of the biopsy device 10. The control buttons may include a plurality of positioning buttons 134, a prime/pierce button 136, a sample button 138, or the like. The control buttons may further include a vacuum button 140, which may be configured to control the amount of vacuum applied to the biopsy device 10. The visual indicators may include one or more light emitting diodes. The aural indicator may include a buzzer or other audible feedback device (e.g., a speaker). In some embodiments, the control buttons may further provide tactile feedback to the user when activated (e.g., haptic feedback). It is noted that while control buttons are indicated, the buttons may instead be any combination of user input devices such as, but not limited to, buttons, toggles, switches, slides, touch screens, etc.

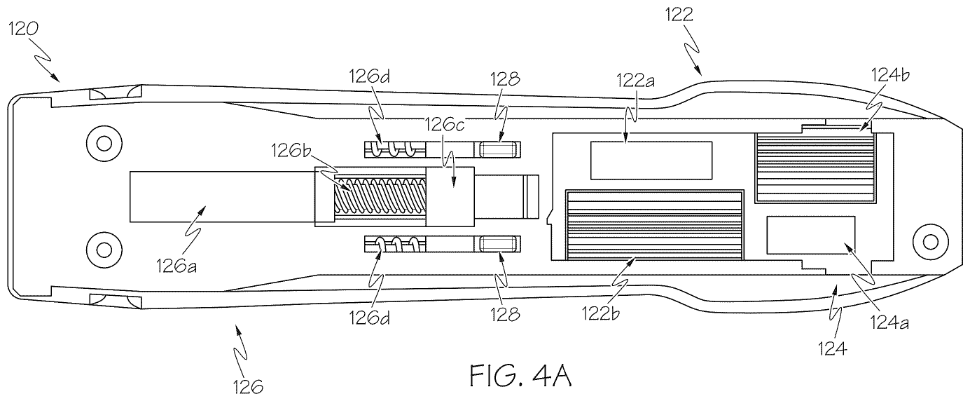

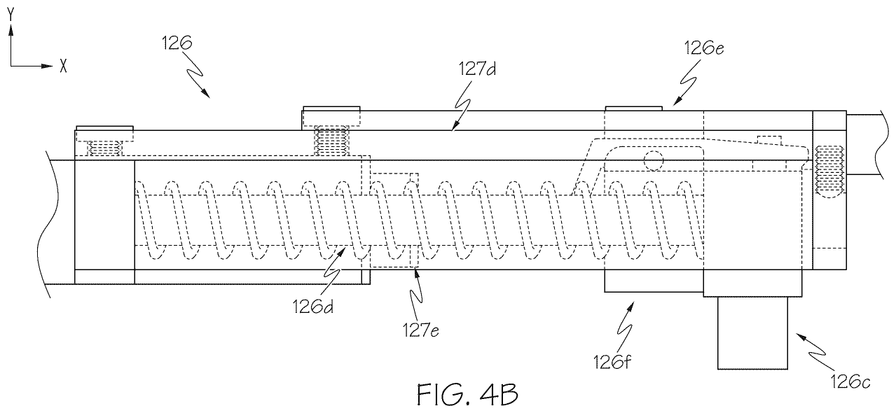

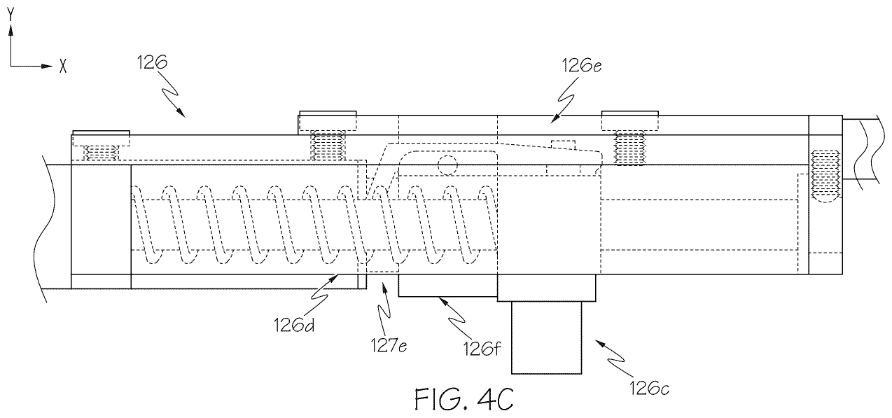

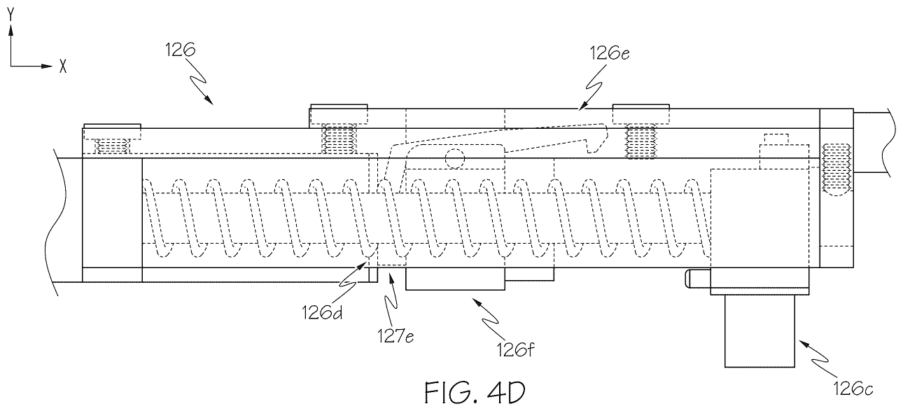

Referring specifically to FIG. 4A, an underside (or coupling side) of the biopsy driver assembly 100 is generally depicted. The biopsy driver assembly 100 may include a plurality of modules (e.g., structures, gears, motors, etc.) for mechanically driving one or more functions of the biopsy device. For example, the biopsy driver assembly 100 may include capital equipment, which may include, for example, a cutter module 122, a rotation module 124, and a piercing module 126. The cutter module 122 may include an electrical motor 122a having a shaft to which a drive gear 122b is attached. The rotation module 124 may include an electrical motor 124a having a shaft to which a drive gear 124b is attached. Piercing module 126 may include an electrical motor 126a, a drive spindle 126b, a carriage 126f, and a piercing drive 126c. Each electrical motor 122a, 124a, 126a may be, for example, a direct current (DC) motor or stepper motor. As an alternative, to the arrangement described above, each of the cutter module 122, rotation module 124, and piercing module 126 may include one or more of a gear, gear train, belt/pulley arrangement, etc., interposed between the respective motor and drive gear or drive spindle. As depicted, in embodiments, openings may be formed within the driver housing 110 to allow to coupling to the biopsy probe assembly 200.

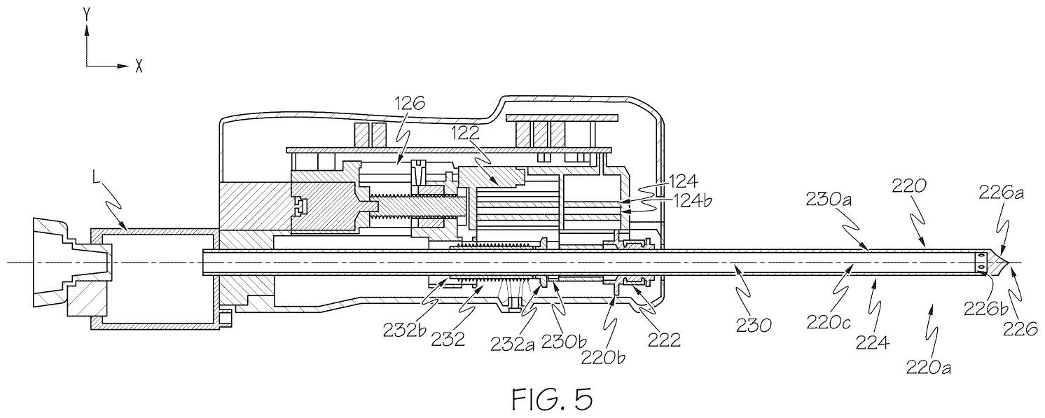

Operation of the piercing module 126 may be most clearly illustrated in FIGS. 4B-4D. As shown in FIG. 4B, the piercing module 126 is illustrated prior to firing. It is noted that the piercing module 126 when assembled to the biopsy probe assembly 200 is operable to pull one or more components of the biopsy probe assembly 200 in a proximal direction (e.g., in the −x direction as depicted in the coordinate axis of FIG. 4B) and fire the biopsy probe assembly 200 into a target site. For example, and with reference to FIG. 5, the piercing drive 126c may engage one or more portions of the biopsy probe assembly 200 connected to the sample notch cannula (described in greater detail herein), to retract and drive forward the sample notch cannula. The piercing drive 126c, accordingly, may be any structure which may extend into and engage one or more portions of the biopsy probe assembly 200 for retracting and firing the sample notch cannula. For example, the piercing drive 126c may include one or more projections (e.g., forks), such as two projections, which are configured to extend into the biopsy probe assembly 200 to the operably engaged to the sample notch cannula. Referring now to FIG. 4B, the piercing drive 126c may be positioned in a distal position of the piercing module 126 when the piercing module is not primed (e.g., not withdrawn in a proximal direction in preparation for firing).

In some embodiments, the carriage 126f may be mounted to the drive spindle 126b (depicted in FIG. 4A). Rotation of the drive spindle 126b via the electrical motor 126a causes the carriage 126f to retract in a proximal direction. It should be noted that, in some embodiments, the drive spindle 126b may take the form of a ball screw, or other similar mechanism. In these embodiments, the ball screw may require less torque from the electrical motor 126a to retract the carriage 126f in the proximal direction, thereby increasing the efficiency of the piercing module 126.

Referring still to FIGS. 4B-4D, mounted to the carriage 126f may be one or more latches 126e which may be moveable between an engaged position and a disengaged position. In other embodiments, the latches 126e may be mounted to a stationary frame of the piercing module 126. In these embodiments, rotation of the drive spindle 126b via electrical motor 126a may cause the carriage 126f to retract in a proximal direction (e.g., in the −x direction as depicted in the coordinate axes of FIGS. 4B-4D) until the piercing drive 126c contacts the one or more latches 126e. As the piercing drive 126c retracts, the contact between the piercing drive 126c and latches 126e may cause the latches 126e to move to the engaged position. In the engaged position illustrated in FIGS. 4B and 4C, the one or more latches engage an edge or other retention feature of the piercing drive 126c. Accordingly, when the one or more latches 126e are engaged with the piercing drive 126c, proximal movement of the carriage 126f pulls the piercing drive 126c proximally, which causes the sample notch cannula to be pulled proximally.

In these embodiments, the piercing drive 126c may translate by sliding along one or more guides 127d. For example, the one or more guides 127d may include a first guide and a second guide arranged parallel to and on either side of the drive spindle 126b. Around each guide may be a firing spring 126d, which compresses as the piercing drive 126c is withdrawn proximally. The carriage 126f may be withdrawn until the one or more latches 126e engage an actuating surface 127e (e.g., such as a backstop) which causes the one or more latches 126e to move or rotate to the disengaged position, shown in FIG. 4D. In the disengaged position, the one or more firing springs 126d are allowed to extend distally (e.g., in the +x direction as depicted in the coordinate axis of FIG. 4D) to fire the piercing drive 126c in the distal direction. As the piercing drive 126c is operably coupled to the sample notch cannula, the sample notch cannula is also fired forward to a desired target site. This firing of the sample notch cannula may be particularly useful when biopsying dense tissue, specifically where manual or slow advancement of the sample notch cannula would be difficult due to variations in tissue type and/or density.

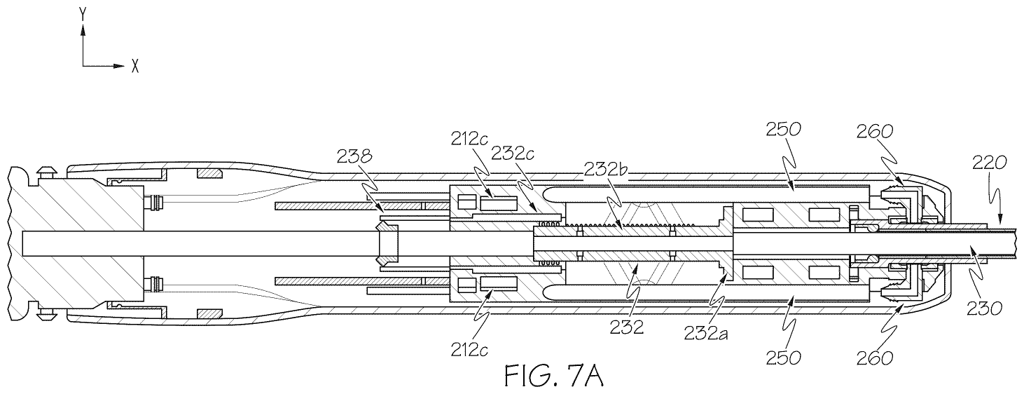

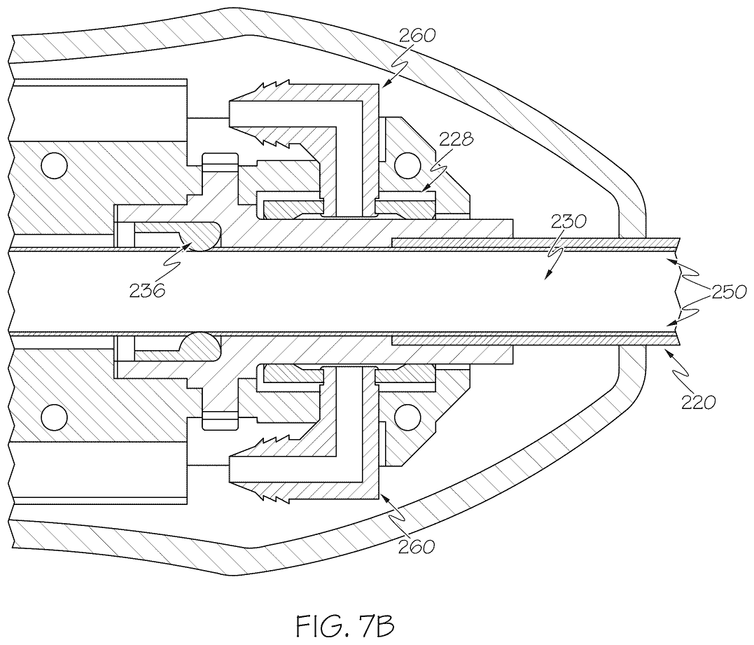

To facilitate coupling of the biopsy driver assembly 100 to the biopsy probe assembly 200 such that the prime-pierce operation described above may be achieved, the biopsy probe assembly 200 may include one or more openings for receiving the one or more projections of the piercing drive 126c. As illustrated in FIG. 7A, the probe assembly may include a probe housing 210 having piercing module slots 212c, each of which may be configured, e.g., in size and in shape, to receive the one or more projections of piercing drive 126c. By engaging piercing module slots 212c with the one or more projections piercing drive 126c, it may be possible to ensure that the probe housing 210 is aligned with the piercing module so as to effect longitudinal movement (e.g., movement in the +/−direction as depicted in the coordinate axis of FIG. 7A) of the sample notch cannula 220 in unison with longitudinal movement of piercing drive 126c during a piercing shot (firing) operation.

While the present embodiment includes two piercing module slots for symmetry and/or redundancy, an alternative embodiment may have, for example, only one piercing module slot. In embodiments, positioned within the biopsy probe assembly 200 may be a second carriage, which is operably coupled to the sample notch cannula 220 and/or other structures within the probe housing 210. The second carriage may be slidingly mounted to one or more rails to allow the second carriage to be slid in proximal and distal directions. In some embodiments, the piercing module slots 212c are formed directly into the second carriage to allow the one or more projections of the piercing drive 126c to engage directly with the second carriage.

During the prime/pierce operation according to the present embodiment, rotation of drive spindle 126b of piercing module 126 by motor 126a (see FIG. 4A) results in a linear translation of carriage 126f in a proximal direction. With the one or more latches 126e in the engaged position, proximal movement of the carriage 126f pulls the piercing drive 126c proximally, which causes the sample notch cannula (via interaction with the second carriage, for example) and piercing drive 126c to be translated in unison in the proximal direction to position piercing drive 126c and sample notch cannula in the ready, i.e., cocked (primed) position where firing springs 126d are compressed. Upon second actuation of prime/pierce button 136 (or operation of a separate actuator) to effect a piercing shot, the carriage 126f is moved further in the proximal direction such that the one or more latches 126e abuts the actuating surface 127e. Contact between the one or more latches 126e and actuating surface 127e may cause the one or more latches 126e to transition to the disengaged position, allowing the firing springs 126d to rapidly propel the sample notch cannula and piercing drive 126c in unison in the distal direction such that the sample notch cannula pierces tissue within the patient.

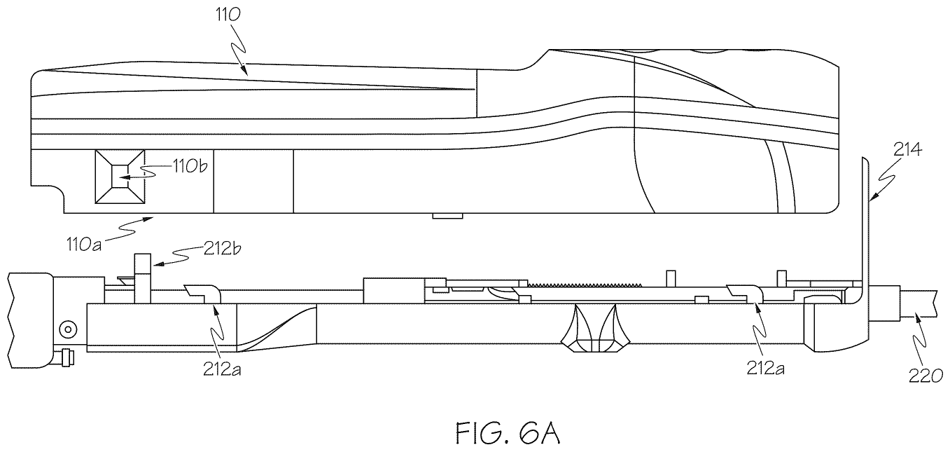

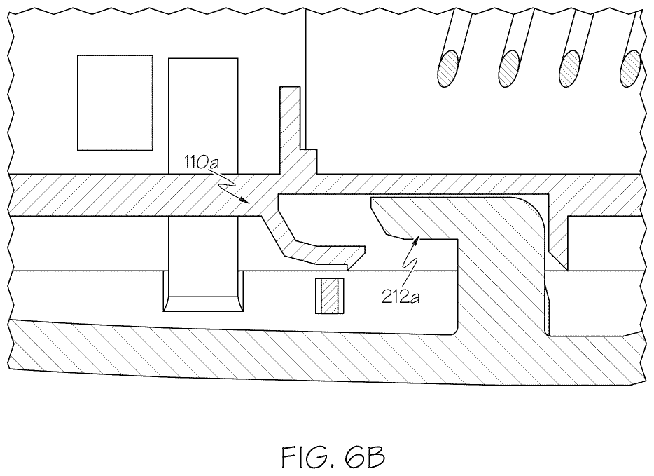

Turning now to FIGS. 5, 6A and 6B, a cross-sectional view of the biopsy device 10 is illustrated. In these embodiments, the biopsy probe assembly 200 may include the probe housing 210, the sample notch cannula 220, a sample notch gear 222, a cutter cannula 230, and a cutter gear-spindle set 232 for rotary and linear cutter translation. In the embodiments described herein, rotational movement of the cutter gear-spindle 232 may result in longitudinal movement of the cutter cannula in a proximal or distal direction.

As most clearly seen in FIG. 6A, the probe housing 210 may be formed as an L-shaped structure having an elongate portion 212 and a front plate 214. The elongate portion 212 may further comprise a plurality of retaining hooks 212a, which may be configured to engage with a plurality of slots 110a in the driver housing 110 (as illustrated in FIG. 6B). When the biopsy probe assembly 200 is attached to biopsy driver assembly 100, the biopsy driver assembly 100 may be initially positioned above the biopsy probe assembly 200 such that the plurality of slots 110a of the driver housing 110 are proximally offset in a longitudinal direction. In some embodiments, the biopsy driver assembly 100 and biopsy probe assembly 200 may be offset between a distance of about 0.1 to about 10 mm, such as about 5 mm. With the plurality of retaining hooks 212a of the probe housing 210 properly offset, the biopsy driver assembly 100 may then be lowered onto the biopsy probe assembly 200 until the probe housing 210 and driver housing 110 contact. The biopsy driver assembly 100 may then be pushed distally with respect to the biopsy probe assembly 200, such that a front face of the driver housing 110 engages the front plate 214 of the probe housing 210 and the plurality of retaining hooks 212a lock into the plurality of slots 110a of the driver housing 110. In this configuration, the front plate 214 of the probe housing 210 may shield the entirety of the front face of the non-disposable driver assembly 100 from contact with a patient.

In an alternate embodiment, the biopsy driver assembly 100 may be initially positioned above the biopsy probe assembly 200 such that a front surface of the biopsy driver assembly 100 is aligned with the front plate 214 of the biopsy probe assembly 200. Once the biopsy driver assembly 100 is appropriately positioned, the front surface of the biopsy driver assembly 100 may be lowered such that the surface comes into contact with the biopsy probe assembly 200. A back end of the biopsy assembly may then be lowered onto the biopsy probe assembly 200 to lock the biopsy driver assembly 100 to the biopsy probe assembly 200. In these embodiments, the biopsy probe assembly 200 may further include at least one tab 212b and the biopsy driver assembly 100 may include at least one notch 110b for receiving the at least one tab 212b of the biopsy probe assembly 200. The engagement of the at least one tab 212b with the at least one notch may act to lock the biopsy driver assembly 100 to the biopsy probe assembly 200. In embodiments, the biopsy driver assembly 100 may be coupled to the biopsy probe assembly 200 using both the plurality of retaining hooks 212a and the at least one tab 212b.

Referring collectively to FIGS. 5-6B, the sample notch cannula 220 may include a proximal portion 220b and distal portion 220a. Referring specifically to FIG. 5, the distal portion 220a may include a sample notch 224. Attached to distal portion 220a is a piercing tip 226, which may forms part of the sample notch cannula 220. The sample notch 224 is formed as an elongate opening in a side wall 220c of the sample notch cannula 220 to facilitate reception of tissue into a lumen of the sample notch cannula 220. The sample notch 224 may extend in a longitudinal direction along longitudinal axis L. In embodiments, the sample notch 224 may not extend in side wall 220c below a centerline of the diameter of the sample notch cannula 220, and may include cutting edges around the perimeter of the opening formed by the sample notch 224.

The sample notch cannula 220 may be rotated about its axis by activation of the rotation module 124, discussed above. For example, when the biopsy probe assembly 200 and the biopsy driver assembly 100 have been attached, drive gear 124b may engage the sample notch gear 222 such that rotation of the drive gear 124b in turn rotates the sample notch gear 222. In these embodiments, the sample notch cannula 220 may be rotated to a plurality of angular positions by sample notch gear 222 in order to allow the biopsy device 10 to obtain tissue from a plurality of target sites around sample notch cannula 220 without requiring the user to manually rotate the position of biopsy driver assembly 100.

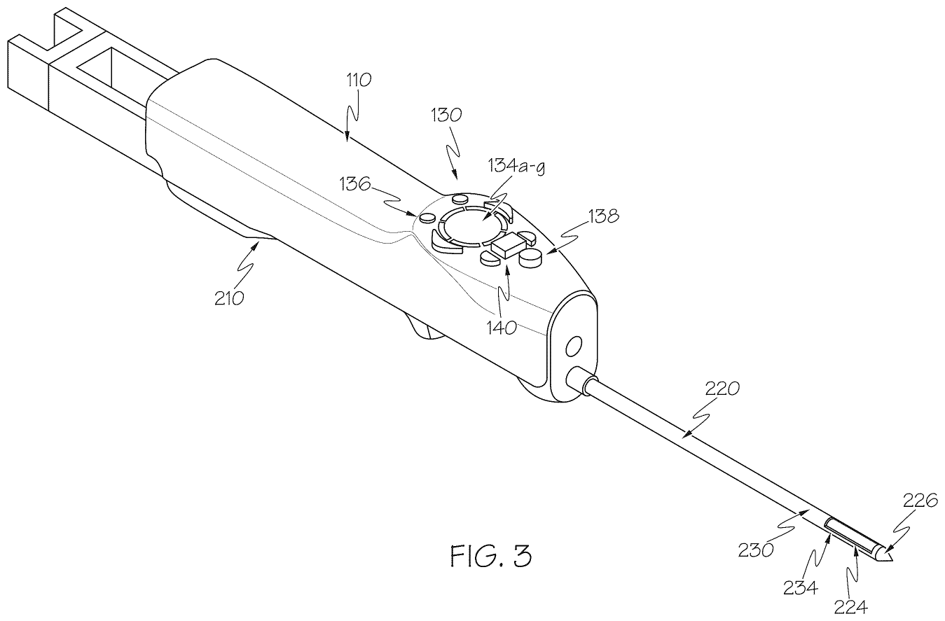

Referring again to FIG. 3, the rotational position of the sample notch cannula 220 may be adjusted by a user using the positioning buttons 134a-f of the controller 130 of the driver housing 110. For example, the controller 130 illustrated in FIG. 3 includes six positioning buttons 134, each of which correspond to a rotational position of the sample notch cannula 220. When a user activates any of the positioning buttons 134, the sample notch cannula 220 is rotated to correspond with the activated positioning button 134. For instance, when positioning button 134b is activated, the rotation module 124 may rotate the sample notch cannula 220 about its axis 60 degrees in a clockwise direction, such that the sample notch 224 is exposed to tissue positioned proximal to the sample notch cannula 220 at 60 degrees from the original position. If positioning button 134f is activated next, the rotation module may rotate the sample notch cannula 220 about its axis 120 degrees (compared to the position corresponding with positioning button 134b) in a counterclockwise direction, such that the sample notch 224 is exposed to tissue positioned proximal to the sample notch cannula 220 at the new position. In these embodiments, the sample notch cannula 220 may be rotated as many times as is necessary to obtain a desired number of tissue samples. Although the biopsy device illustrated in FIG. 3 shows the controller 130 comprising six positioning buttons, it should be understood that the controller 130 may comprise a greater or fewer number of positioning buttons, each of which may correspond with a rotational position of the sample notch cannula 220. In other embodiments, the controller 130 may only comprise two buttons, wherein a first button rotates the sample notch cannula 220 a predetermined amount in a first, or clockwise, direction and a second button rotates the sample notch cannula in a reverse, or counterclockwise, direction. In some embodiments, the positioning buttons 134 may illuminate (e.g., via an integrated lighting device (e.g., light bulb, LED, etc.) when selected by a user, such that the user is able to easily see the corresponding position of the sample notch cannula 220.

Referring now to FIG. 5, the piercing tip 226 of the sample notch cannula 220 may include a tip portion 226a and a mounting portion 226b. In some embodiments, the piercing tip 226 is inserted into the sample notch cannula 220 at distal portion 220a, with the mounting portion 226b of the piercing tip 226 being attached to the distal portion 220a of the sample notch cannula 220, such as via an adhesive or weld. As such, the tip portion 226a of the piercing tip 226 may extend distally from the distal portion 220a of the sample notch cannula 220. In some embodiments, the tip portion 226a may be integral with the sample notch cannula 220.

Referring still to FIG. 5, cutter cannula 230 may be positioned coaxially within the sample notch cannula 220 and may include a proximal portion 230b and a distal portion 230a. The distal portion 230a may further include a cutting edge 234 for severing a tissue sample. The cutter gear-spindle set 232 may be fixedly attached (e.g., glued, welded, staked, etc.) to the proximal portion 230b of the cutter cannula. The cutter gear-spindle set 232 may be a unitary device having a driven gear 232a fixedly attached to a threaded spindle 232b. In some embodiments, the cutter gear-spindle set 232 may be formed as a single molded component, while in other embodiments, the components may be formed separately. The cutter cannula 230 may be retracted or extended along a longitudinal axis L by activation of cutter module 122 of biopsy driver assembly 100, with the drive gear 122b of the cutter module 122 engaging the driven gear 232a of the cutter gear-spindle set 232. Thus, cutter cannula 230 has a rotational cutting motion and is translated axially along the longitudinal axis L. In these embodiments, the pitch of the threads of threaded spindle 232b may determine the number of revolutions per axial distance that cutter cannula 230 moves axially.

As further illustrated in FIG. 5, the sample notch cannula 220 and cutter cannula 230 may be coaxially arranged along the longitudinal axis L in a nested tube arrangement, with cutter cannula 230 being the innermost tube and sample notch cannula 220 being the outermost tube. The sample notch cannula 220 and cutter cannula 230 may be coaxially arranged such that a space exists between the two cannulas.

In some embodiments, this space may define a fluid pathway 250, which may allow a fluid, such as air, saline, or anesthetics, to pass between the sample notch cannula 220 and the cutter cannula 230, as may be further illustrated in FIGS. 7A and 7B. In some embodiments, the fluid pathway 250, which passes between the sample notch cannula 220 and the cutter cannula 230, may function as a vent pathway to facilitate severed tissue being pulled from the region of the sample notch 224 and moved axially through cutter cannula 230 of the biopsy device 10 to the sample collection region.

In one mode of operation, when the vacuum is removed from cutter cannula 230, a fluid may be injected into the fluid pathway 250, such that the fluid which passes between the sample notch cannula 220 and the cutter cannula 230 may be discharged into tissue by way of the sample notch 224.

In embodiments in which a vacuum is created within the biopsy device 10, vacuum may be maintained within the biopsy device 10 by a series of seals. As illustrated in FIG. 7B, sample notch seal 228 may act to seal the sample notch cannula 220 to the sample notch gear 222. Similarly, seal 236 may act to seal the cutter cannula 230 to the sample notch cannula 220. Although the seal 236 and sample notch cannula 220 are depicted as being separate components, in some embodiments, the seal 236 and the sample notch cannula 220 may be formed as a single, integral component. By forming the seal 236 integrally with the sample notch cannula 220, the integrity of the seal between the sample notch cannula 220 and the cutter cannula 230 may be improved.

Referring still to FIGS. 7A-7B, in order to allow fluid to enter the fluid pathway 250, a tube connector 260 may be scaled to the sample notch cannula 220. In these embodiments, the tube connector 260 may be formed as a single, monolithic structure, which may in turn be scalably coupled to internal tubing (not shown). The internal tubing may be connected to a vacuum source for supplying a vacuum to the biopsy device 10 and/or a syringe (or pump) for supplying anesthetics or medication to tissue at a biopsy site. Although the tube connector 260 depicted in FIG. 7B is illustrated at a single component, it should be understood that, in some embodiments, the tube connector 260 may include a plurality of tube connectors, such as a first tube connector and a second tube connector, which may be formed as separate components. In embodiments in which the tube connector 260 includes the plurality of tube connectors, each of the plurality of tube connectors may be sealably coupled to internal tubing and connected to the vacuum source for supplying vacuum to the biopsy device.

Furthermore, although the sample notch seal 228 and the tube connector 260 are depicted as being separate components, in some embodiments, the sample notch seal 228 and the tube connector 260 may be formed as a single, integral component. By forming the tube connector 260 integrally with the sample notch seal 228, the integrity of the seal between the sample notch cannula 220 and the sample notch gear 222 may be improved.

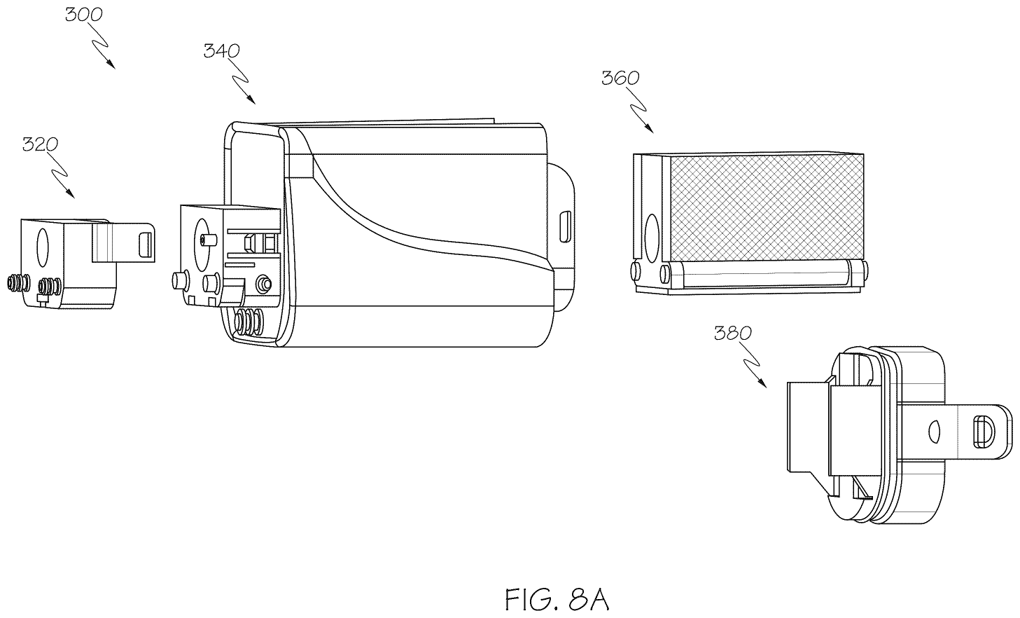

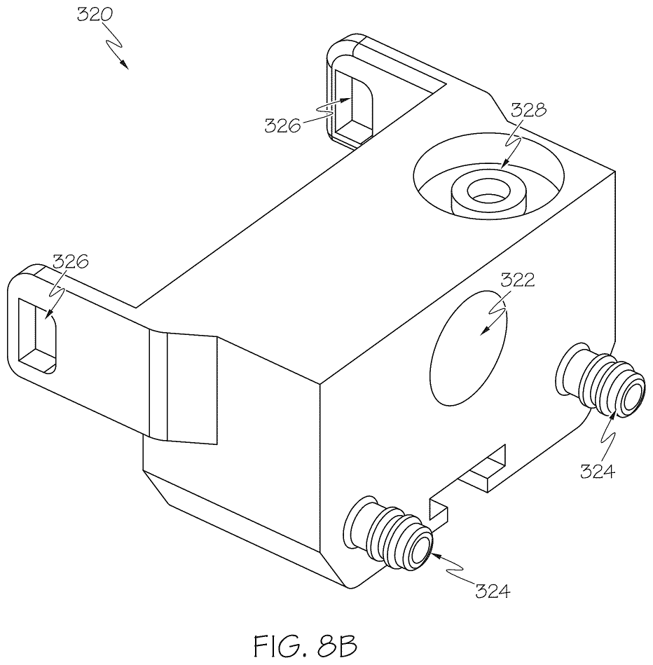

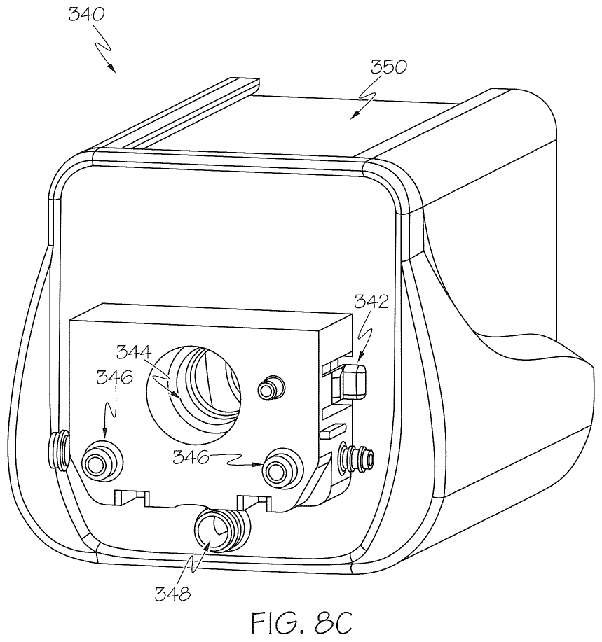

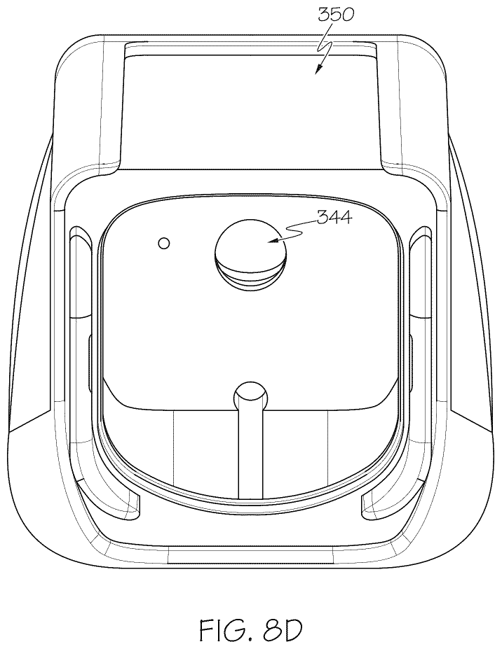

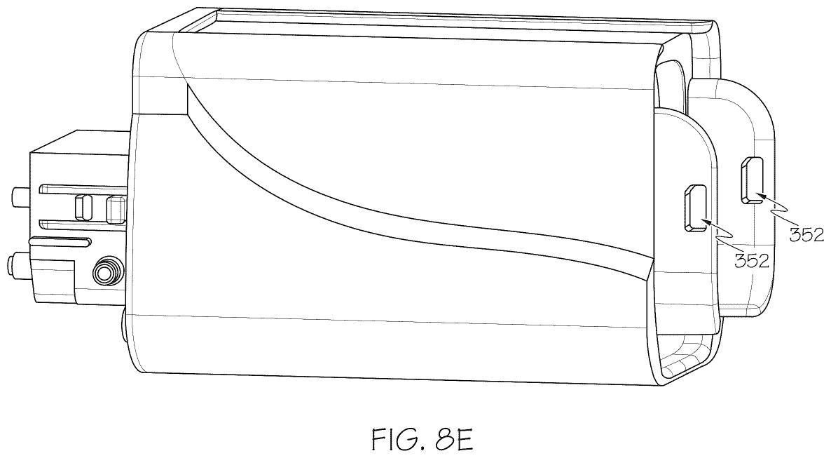

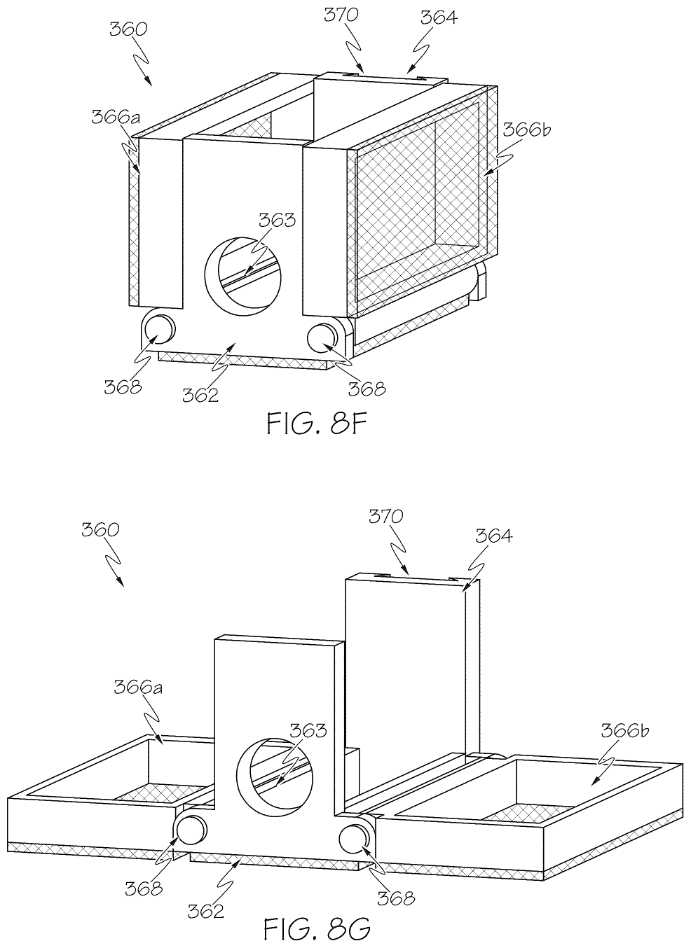

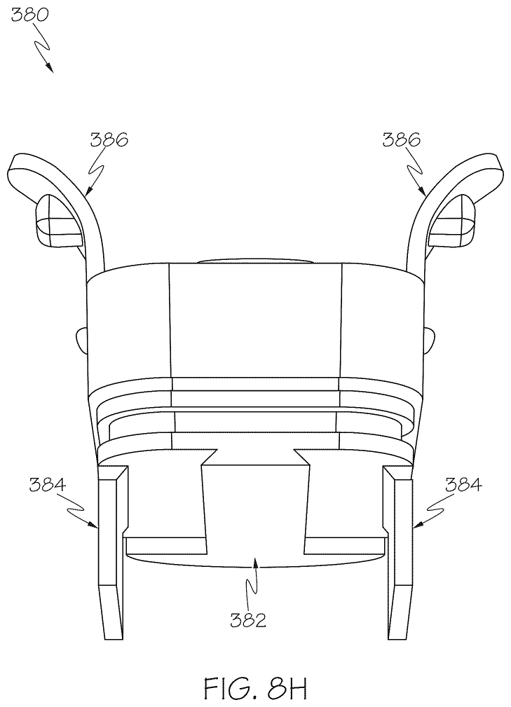

Turning now to FIGS. 8A-8H, the biopsy device may further comprise sample basket assembly 300. The sample basket assembly 300 may include a probe interface 320, an outer basket housing 340, a hinge basket assembly 360 and a rear housing 380. The hinge basket assembly 360 may be mounted to the probe housing 210 and receive tissue samples via the cutter cannula 230. For example, the sample basket assembly 300 may be mounted to a distal end of the probe housing 210, such as illustrated in FIG. 2.

As most clearly illustrated in FIG. 8B, the probe interface 320 includes a cutter opening 322 which may be configured to receive the proximal portion 230b of the cutter cannula 230. In some embodiments, the fit between the proximal portion 230b of the cutter cannula 230 and the cutter opening 322 may create a sealed pathway for the cutter cannula 230. As further illustrated in FIG. 8B, the probe interface 230 may further include barbed mechanisms 324, which may be configured to couple with a venting line and/or a saline line for providing venting, saline, and/or anesthetics to the biopsy device 10. The probe interface 320 may also comprise openings 326, which may be configured to attach the probe interface 320 to the outer basket housing 340. In some embodiments, the probe interface 320 may further include a vacuum sensor connection 328, which may be configured to receive a vacuum sensor. The vacuum sensor may be a pressure differential sensor or other vacuum sensor that is configured to provide vacuum feedback signals to a biopsy device station 500. The vacuum sensor may determine whether the vacuum pressure within the biopsy device 10 deviates by more than a predetermine amount from a baseline vacuum pressure during operation of the biopsy device 10. For example, if the vacuum pressure falls below the baseline vacuum pressure by more than an allowable deviation, this may indicate an incomplete seal which may lead to a warning issued by the biopsy device station 500 to check the probe is correctly configured for operation. Conversely a higher than expected pressure during tissue transport may indicate a blockage in the sample notch cannula and cause the device to prolong the tissue transport sequence to ensure successful tissue transport. In these embodiments, the vacuum sensor may signal to the biopsy device station 500 that the tissue sample should repeated without user intervention.

Turning now to FIGS. 8C-8E, the outer basket housing 340 may include tabs 342 which engage the openings 326 of the probe interface 320. Furthermore, the outer basket housing 340 may include a cutter entrance 344, which may be coaxially aligned with the cutter opening 322 of the probe interface 320 when the two components are attached. The outer basket housing 340 may further include port connections 346, which may be configured to connect the biopsy device to the venting line and/or the saline line.

Additionally, the outer basket housing 340 may comprise vacuum fitting 348, which may be configured to connect the biopsy device 10 to a vacuum source. In these embodiments, the biopsy device station 500 may include a vacuum source, which may be configured to build up negative pressure within the biopsy device 10. The biopsy device station 500 may further comprise a saline or anesthetic pump, which may be configured to provide saline and/or anesthetics to the biopsy device 10.

As most clearly illustrated in FIG. 8D, the outer basket housing 340 may further comprise a recess 350 positioned in a top surface of the outer basket housing 340. In some embodiments, the recess 350 may be configured to receive a magnifying glass, which may enable a user to more clearly view the contents of the hinge basket assembly 360 without needing to remove tissue samples from the hinge basket assembly 360. Furthermore, FIG. 8E illustrates that the outer basket housing 340 may include openings 352 configured to couple the outer basket housing 340 to the rear housing 380.

Turning now to FIG. 8F, the hinge basket assembly 360 is shown. The hinge basket assembly 360 may include a front wall 362 and a back wall 364 opposing the front wall 362. Furthermore, the hinge basket assembly may include a first side wall 366a opposing a second side wall 366b. In some embodiments, the first and second side walls 366a,b may comprise a mesh material, which may allow for fluid drainage and may assist in the retention of tissue samples. Additionally, the mesh material may maximize x-ray contrast for sample radiographs.

As further illustrated in FIG. 8F, the front wall 362 may comprise a cutout 363, which may be configured to receive the cutter cannula 230. When the hinge basket assembly 360 is connected to the outer basket housing, the cutout 363 may be coaxially aligned with the cutter opening 322 and the cutter entrance 344. The first and second side walls 366a,b may be connected to the front wall 362 and back wall 364 by way of hinges 368. Although FIG. 8F illustrates the first and second side walls 366a,b in a closed configuration, the hinges 368 may allow the first and second side walls 366a,b to rotate about their axis to an open position, as more clearly shown in FIG. 8G. The back wall 364 of the hinge basket assembly 360 may also include a slot 370 which may be configured for connecting the hinge basket assembly 360 to the rear housing 380.

Turning now to FIG. 8H, the rear housing 380 is illustrated. The rear housing 380 may include connection interface 382, which may be configured to engage the slot 370 of the hinge basket assembly 360. When the connection interface 382 of the rear housing 380 is secured to the slot 370 of the hinge basket assembly 360, the rear housing 380 may act to seal the hinge basket assembly 360. Furthermore, the rear housing 380 may comprise side walls 384, which may be configured to maintain the first and second side walls 366a,b of the hinge basket assembly 360 in the closed position. In these embodiments, when the hinge basket assembly 360 is detached from the rear housing 380, the first and second side walls 366a,b may be capable of rotating about hinges 368 to the open configuration.

The rear housing 380 further includes tabs 386, which may be configured to engage the openings 326 of the outer basket housing 340. When the tabs 386 of the rear housing 380 are engaged with the openings 326 of the outer basket housing 340, the hinge basket assembly 360 may be effectively sealed between the rear housing 380 and the outer basket housing 340.

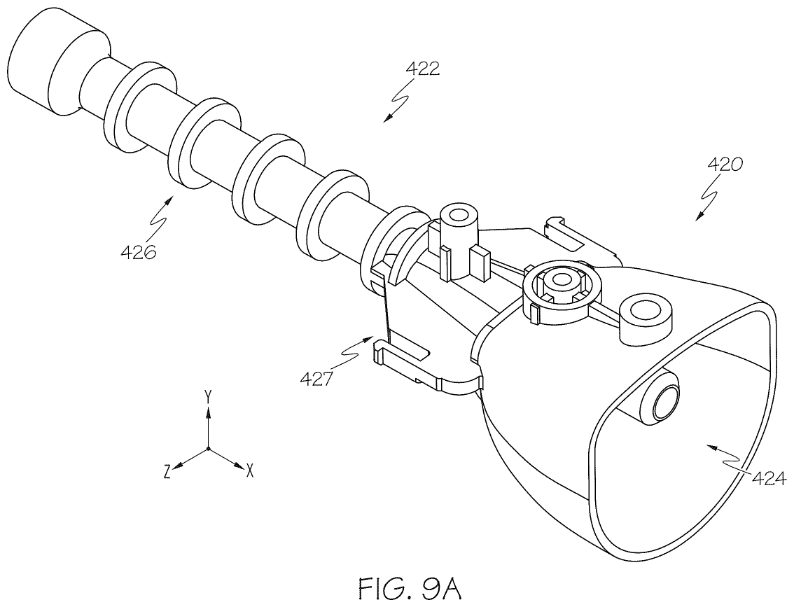

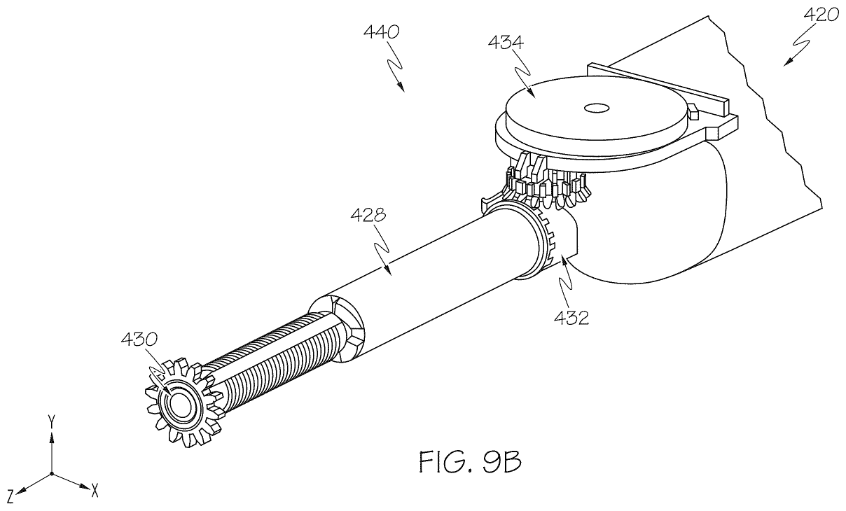

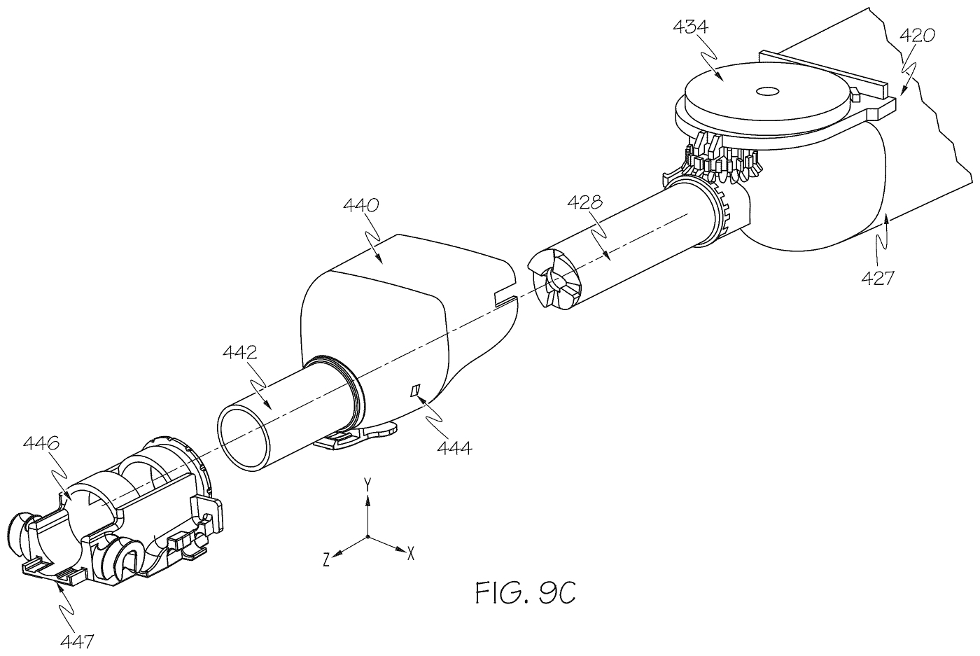

Referring now to FIGS. 9A-13B, the biopsy device 10 may be further configured to deliver a marker to a target site. In these embodiments, the biopsy device 10 may further comprise a marker delivery assembly 400. As shown in FIGS. 9A-13B, the marker delivery assembly 400 may include an integration module 420 and a marker delivery module 450, as will be described in additional detail herein. In these embodiments, the integration module 420 may act as an interface between the biopsy probe assembly 200 and the marker delivery module 450. The marker delivery module 450 may act as an interface between the integration module 420 and the sample basket assembly 300, and may be configured to deliver a marker to a target site via the integration module 420.

Referring now to FIGS. 9A-9C, the integration module 420 of the marker delivery assembly 400 is depicted. As shown in FIG. 9A, the integration module 420 may comprise a connector 422 having a cavity 424. The connector 422 may further include a neck 426, which may extend outwardly from the cavity in a longitudinal direction. In these embodiments, the neck 426 may be inserted within the biopsy probe assembly 200 in order to releasably couple the marker delivery assembly 400 to the biopsy probe assembly 200. In some embodiments, the neck 426 may include a plurality of rings, which may increase the structural rigidity of the connector 422. As further depicted in FIG. 9A, the neck 426 may further include a plurality of clips 427 for securing the neck 426 to additional components of the integration module 420, as will be described in additional detail herein. In these embodiments, the cavity 424 may be configured to receive a housing of the marker delivery module 450, as will be described in additional detail herein.

Referring now to FIG. 9B, the integration module 420 may further include a transmission cover 440, which may include a shaft 428, such as a hollow shaft, and a bevel drum 434. In these embodiments, the shaft 428 may be coaxially disposed about the neck 426 of the integration module 420, such that the shaft 428 is capable of rotating about the neck 426. Accordingly, it should be understood that the hollow portion of the shaft 428 may have the same shape as the neck 426, such that the neck 426 may receive the shaft 428. For example, as depicted in FIGS. 9A and 9B, the neck 426 and the shaft 428 may both have a cylindrical shape, although it should be understood that the neck 426 and the shaft 428 may take any shape without departing from the scope of the present disclosure.

Referring still to FIG. 9B, the shaft 428 may include a distal end having a spindle gear 430 and a proximal end having a drum gear 432. In these embodiments, the spindle gear 430 may engage the spindle 232b of the biopsy probe assembly 200 when the integration module 420 is coupled to the biopsy probe assembly 200, such that rotation of the spindle 232b further drives the spindle gear 430, causing the shaft 428 to rotate.

Referring still to FIG. 9B, the drum gear 432 located on the proximal end of the shaft 428 may engage the bevel drum 434, such that rotation of the drum gear 432 results in similar rotation of the bevel drum 434. Because the drum gear 432 and the spindle gear 430 are both mounted on the shaft 428, rotation of the spindle gear 430 causes the shaft to rotate about a longitudinal axis. As the shaft 428 rotates, the drum gear 432 may rotate along with the shaft 428, such that rotation of the shaft 428 is translated to the bevel drum 434. In these embodiments, the rotation of the bevel drum 434 may cause a pushrod to unwind and force a marker through the biopsy probe assembly 200, as will be described in additional detail herein.

Turning now to FIG. 9C, the integration module 420 may further include a transmission cover 440 having a body 442, a plurality of notches 444, and an adapter 446, such as an inlet adapter. In these embodiments, the body 442 may have a hollow interior portion, such that the body 442 may be coaxially disposed about the shaft 428 of the integration module 420. In these embodiments, the body 442 may take the same shape as the shaft 428, such that the body 442 may be disposed about the shaft 428.

As further depicted in FIG. 9C, the transmission cover 440 may further include a retaining mechanism 448, such as a clip, or other similar mechanism, which may be used to releasably couple the transmission cover 440 to the connector 422. When the retaining mechanism 448 engages the connector 422, the plurality of clips 427 of the connector 422 may further engage the plurality of notches 444 of the transmission cover 440 to aid in securing the transmission cover 440 to the connector 422.

Referring still to FIG. 9C, the adapter 446 may be coaxially disposed about an outer surface of the body 442. In these embodiments, the adapter 446 may further include at least one port 447, such as an inlet port, which may be used to couple the integration module 420 to the biopsy probe assembly 200. For example, the at least one port 447 may be configured to engage an inlet, such as a Y-inlet, positioned within the biopsy probe assembly 200, as will be described in more detail herein.

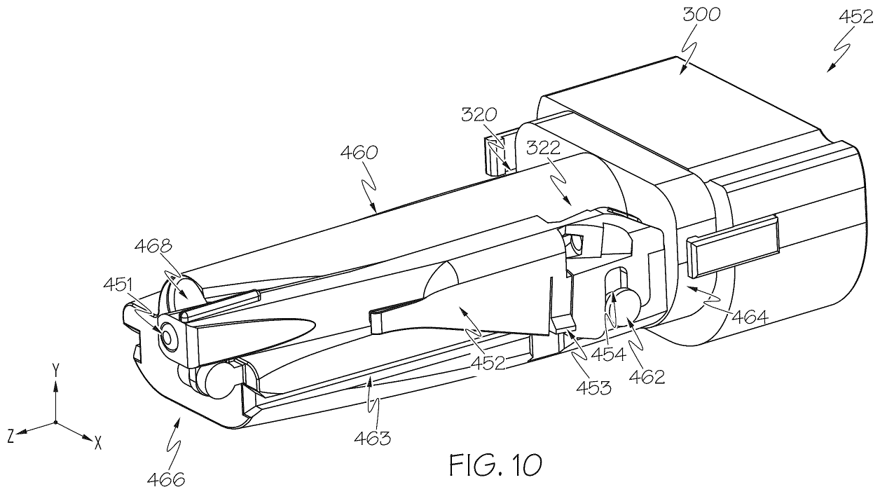

Turning now to FIG. 10, a marker delivery module 450 is depicted. As illustrated in FIG. 10, the marker delivery module 450 may include a marker delivery cartridge 452, which may be rotatably disposed within a marker delivery housing 460. In these embodiments, the marker delivery cartridge 452 may be rotated between an initial position and a delivery position.

The marker delivery cartridge 452 may further include a chamber 451 disposed within the marker delivery cartridge 452. In these embodiments, the chamber 451 may include a plurality of markers, which may be deployed to a target site when the marker delivery assembly 400 is engaged. In these embodiments, the plurality of markers may be pre-loaded into the chamber 451 before the marker delivery cartridge 452 is inserted into the marker delivery housing 460. Additionally, in some embodiments, the plurality of markers may be inserted into the chamber 451 after the marker delivery cartridge 452 is inserted into the marker delivery housing 460, such as by using a pushrod or other similar device.

Referring still to FIG. 10, the marker delivery cartridge 452 may further include a plurality of recesses 454, which may engage at least one coupling rod 462 that may be fixedly positioned within the marker delivery housing 460. In some embodiments, the at least one coupling rod 462 may be integrally formed with the marker delivery housing 460, such that the at least one coupling rod 462 and the marker delivery housing 460 are formed as a single, monolithic structure. The plurality of recesses 454 of the marker delivery cartridge 452 may engage the at least one coupling rod 462 of the marker delivery housing 460, such that the at least one coupling rod 462 releasably secures the marker delivery cartridge 452 in the initial position. For example, in some embodiments, the plurality of recesses 454 may engage in a snap-fit with the at least one coupling rod 462 of the marker delivery housing 460, such that the marker delivery cartridge 452 may be released from the at least one coupling rod 462 when a force is applied to the marker delivery cartridge 452.

As further depicted in FIG. 10, the marker delivery housing 460 may further include at least one slot 463, which may be integrally formed within an interior wall of the marker delivery housing 460. In these embodiments, the marker delivery cartridge 452 may further include at least one latch 453, which may be configured to engage the at least one slot 463 of the marker delivery housing 460 when the marker delivery cartridge is rotated from the initial position to the delivery position. For example, when a force is applied to the marker delivery cartridge 452, the marker delivery cartridge 452 may rotate about the at least one fulcrum rod 461 such that the at least one latch 453 of the marker delivery cartridge 452 engages the at least one slot 463 of the marker delivery housing 460.

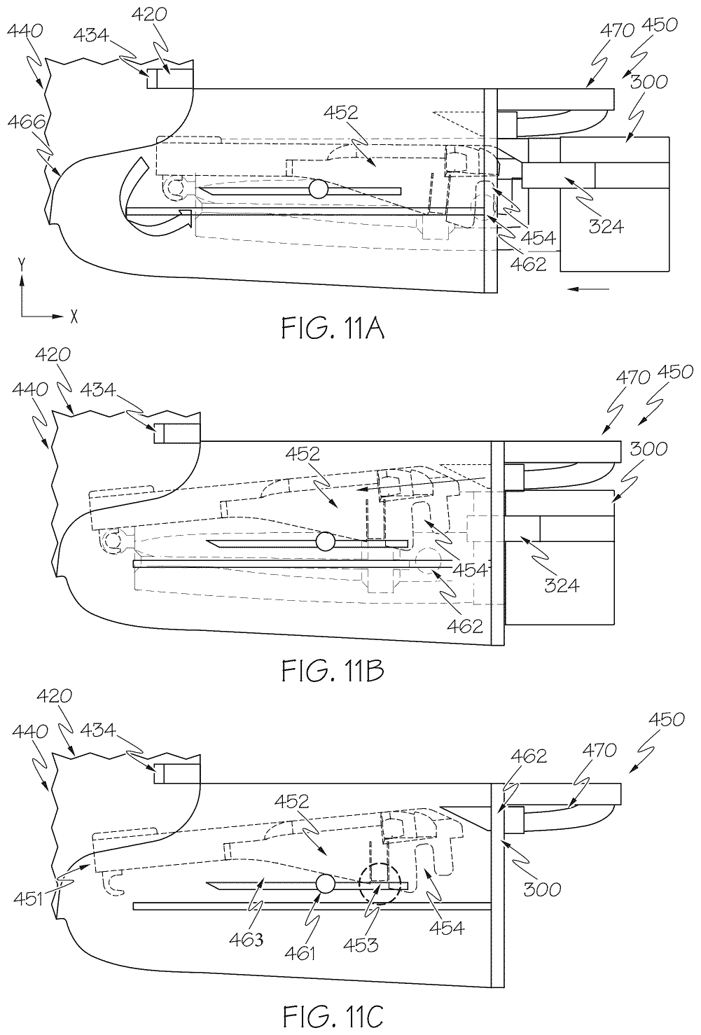

Referring now to FIGS. 10 and 11A-11C, the marker delivery housing 460 may further include a proximal housing interface 464 and a distal end 466. In these embodiments, the proximal housing interface 464 may be adapted to engage the sample basket assembly 300, such that the sample basket assembly 300 is releasably coupled to the marker delivery assembly 400. In these embodiments, the proximal housing interface 464 may be configured to engage the probe interface 320 of the sample basket assembly 300, as is most clearly depicted in FIG. 11A. For example, the proximal housing interface 464 may include a plurality of ports which may receive the barbed mechanisms 324 of the probe interface 320. Additionally, the proximal housing interface 464 may include any mechanism capable of releasably coupling the marker delivery assembly 400 to the sample basket assembly 300.

In these embodiments, the barbed mechanisms 324 may further act to hold open the chamber 451 of the marker delivery cartridge 452, thereby, enabling the pushrod 470 to enter the chamber 451 and force the at least one of the plurality of markers 480 out of the cartridge 452 and into the biopsy probe assembly 200. Because the barbed mechanisms 324 may control access to the chamber 451 of the cartridge 452, a user may ensure that the cartridge 452 and chamber 451 are aligned with the biopsy probe assembly 200 and pushrod 470 to enable at least one of the plurality of markers 480 to be deployed.

Referring still to FIG. 11A, in these embodiments, the distal end 466 may be configured to engage the transmission cover 440 of the integration module 420, such that the marker delivery module 450 is releasably coupled to the integration module 420. In these embodiments, the distal end may be an open distal end, such that components within the marker delivery housing 460 (e.g., marker delivery cartridge 452) may be manipulated via the distal end 466 of the marker delivery housing 460. For example, as the marker delivery module 450 is advanced towards the integration module 420, the transmission cover 440 may contact the marker delivery cartridge 452, such that the marker delivery cartridge 452 rotates about the at least one fulcrum rod 461 from the initial position to the delivery position.

As most clearly depicted in FIG. 10, the marker delivery housing 460 may further include a cutter tube 468. In these embodiments, the cutter tube 468 may be aligned with the cutter opening 322 of the sample basket assembly 300 when the marker delivery housing 460 is coupled to the sample basket assembly 300. Similarly, the cutter tube 468 may be further aligned with the cutter cannula 230 when the marker delivery assembly 400 is coupled to the biopsy probe assembly 200. By aligning the cutter tube 468 with both the cutter cannula 230 and the cutter opening 322, a user may ensure that tissue samples obtained by the biopsy device 10 during a procedure may be transported through the cutter cannula 230 and cutter tube 468 and into the sample basket assembly 300 when the marker delivery assembly 400 is utilized.

Referring now to FIGS. 11A-11C, installation of the marker delivery module 450 into the integration module 420 is depicted. Initially, it should be noted that, in these embodiments, the marker delivery module 450 may further include a pushrod 470, which may be configured to push a marker through the biopsy probe assembly 200 and into a target site. As depicted in FIGS. 11A-11C, the pushrod 470 may be coupled to the bevel drum 434 of the integration module 420. In these embodiments, rotation of the bevel drum 434 may cause the pushrod 470 to unwind, thereby forcing the pushrod 470 and the marker through the biopsy probe assembly 200.

FIG. 11A depicts the marker delivery cartridge 452 in the initial position. With, the marker delivery cartridge 452 in the initial position, the marker delivery module 450 may be aligned with the integration module 420 and advanced in the longitudinal direction towards the integration module 420, such that the marker delivery module 450 engages the integration module 420. More specifically, the marker delivery module 450 is advanced such that the distal end 466 and the marker delivery cartridge 452 contact the transmission cover 440 of the integration module 420.

Referring now to FIG. 11B, as the marker delivery module 450 continues to advance in the longitudinal direction towards the integration module 420, the longitudinal translation of the marker delivery module 450 may cause the transmission cover 440 to apply a downward force (e.g., in the −y direction depicted in the coordinate axis of FIGS. 11A-11C) on the marker delivery cartridge 452. In these embodiments, the downward force applied by the transmission cover 440 on the marker delivery cartridge 452 may increase as the marker delivery module 450 is translated towards the integration module 420. Once a sufficient force has been achieved, the plurality of recesses 454 of the marker delivery cartridge 452 may disengage the at least one coupling rod 462 of the marker delivery housing 460.

Turning now to FIG. 11C, once the plurality of recesses 454 have disengaged the at least one coupling rod 462, the continued downward force applied by the transmission cover 440 to the marker delivery cartridge 452 may cause the marker delivery cartridge to rotate about the at least one fulcrum rod 461, such that the marker delivery cartridge 452 rotates from the initial position to the delivery position. As the marker delivery cartridge 452 rotates about the at least one fulcrum rod 461, the at least one latch 453 of the marker delivery cartridge 452 may engage the at least one slot 463 of the marker delivery housing 460. In these embodiments, the engagement of the at least one latch 453 with the at least one slot 463 may secure the marker delivery cartridge 452 in the delivery position. As depicted in FIG. 11C, the chamber 451 of the marker delivery cartridge 452 may align with the pushrod 470 and the sample notch cannula of the biopsy probe assembly 200 in the delivery position. In these embodiments, as the pushrod 470 unwinds, the pushrod 470 may extend through the chamber 451 of the marker delivery cartridge 452 and the sample notch cannula 220, thereby forcing at least one of the plurality of markers housed within the chamber 451 out of the sample notch cannula 220 and into a target site.

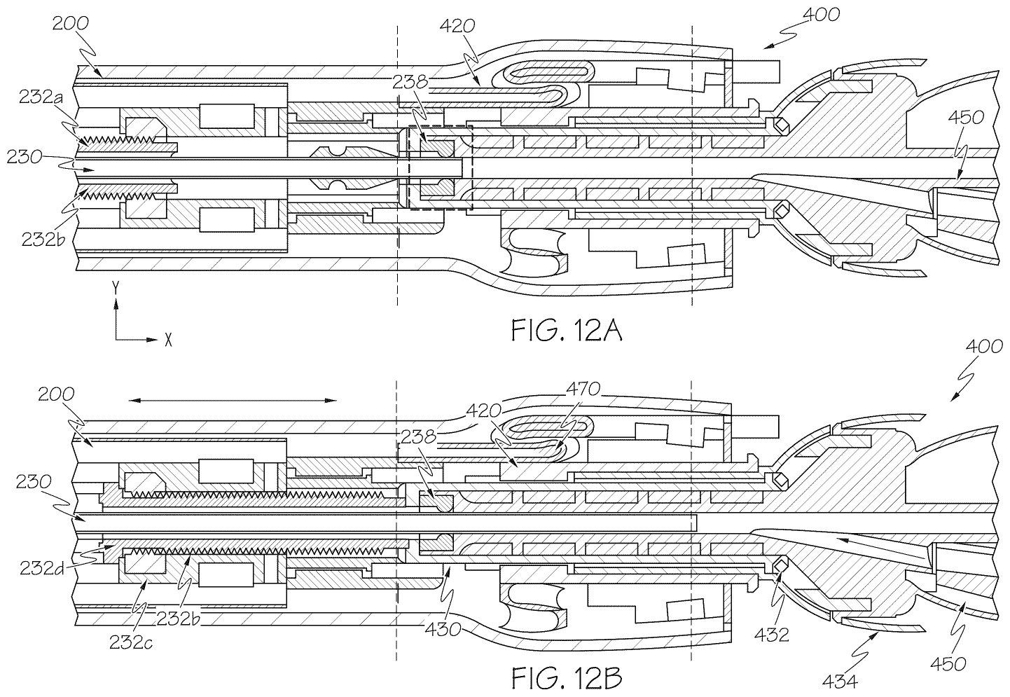

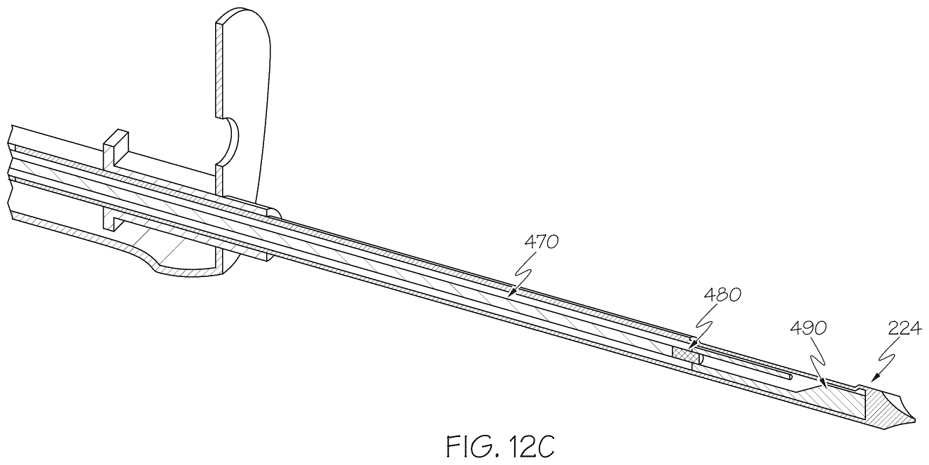

Turning now to FIGS. 12A-12B, the marker delivery assembly 400 is depicted coupled to the biopsy probe assembly 200. As previously discussed herein, in these embodiments, the adapter 446 of the integration module 420 may be coupled to an inlet 238, such as a y-inlet, of the biopsy probe assembly 200 in order to secure the marker delivery assembly 400 to the biopsy probe assembly 200.

In these embodiments, the cutter cannula 230 of the biopsy probe assembly 200 may be moved between an extended position and a fully retracted position, as has been described herein. In the extended position, depicted in FIG. 12A, the spindle 232b of the cutter cannula 230 may not engage the marker delivery assembly 400. For example, in the extended position, the spindle 232b of the cutter cannula 230 may engage the driven gear 232a of the cutter cannula 230, such that the spindle 232b is capable of linearly translating the cutter cannula 230 in an axial direction.

In contrast, FIG. 12B depicts the cutter cannula 230 in the fully retracted position. In the fully retracted position, the cutter gear-spindle 232 may enter a free rotation zone 232d, wherein the external threads of the spindle 232b are disengaged from the internal threads of spindle nut 232c, such that the cutter gear-spindle 232 and cutter cannula 230 may freely rotate without linearly translating the cutter cannula 230 in an axial direction. At this point, the spindle 232b may be free to engage with the spindle gear 430 of the integration module 420 of the marker delivery assembly 400. With the spindle 232b engaged with the spindle gear 430, rotation of the spindle 232b may rotate the spindle gear 430, which in turn may rotate the shaft 428 and drum gear 432 of the integration module 420.

As has been described herein, the bevel drum 434 of the integration module 420 may be in frictional engagement with the pushrod 470. Accordingly, as the drum gear 432 rotates, the bevel drum 434 may unwind the pushrod 470, such that the pushrod 470 extends through the chamber 451 of the marker delivery cartridge 452 and the cutter cannula 230.

As the pushrod 470 moves through the chamber 451, at least one of the plurality of markers 480 positioned within the chamber 451 of the marker delivery cartridge 452 may traverse the cutter cannula 230, as is most clearly depicted in FIG. 12C. The bevel drum 434 may continue to drive the pushrod 470 through the cutter cannula 230 until at least one of the plurality of markers 480 is ejected from the sample notch 224 and into the target site.

Once the marker 480 has been deployed, the biopsy device 10 may be removed from the target site. In some embodiments, the marker delivery module 450 of the marker delivery assembly 400 may further include a ramp 490 which is configured to direct the at least one of the plurality of markers 480 from the sample notch 224 as the pushrod 470 moves the marker 480 to the distal end of the sample notch cannula 220. In these embodiments, the ramp 490 may be loaded into the chamber 451 of the marker delivery cartridge 452 and pushed through the cutter cannula 230, along with marker 480, by the pushrod 470 until the ramp 490 reaches the sample notch 224. Once the ramp 490 has reached the sample notch 224, the continued movement of the pushrod 470 may force the marker 480 to move in a vertical direction up the ramp 490 and out of the sample notch 224.

In the present embodiment, marker delivery assembly 400 does not include any mechanism to re-engage threads of the spindle with the internal threads of the spindle nut. Thus, following marker delivery, the cutter gear-spindle 232 permanently remains in the free rotation zone, which thereby prevents a re-use of the biopsy probe assembly for taking additional biopsy samples. However, in an alternative arrangement, it is contemplated that a biasing mechanism, e.g., spring, may be included to exert a distal force on cutter gear-spindle 232 so as to re-engage the threads of the spindle with the internal threads of the spindle nut, so as to facilitate an additional sample collection cycle.

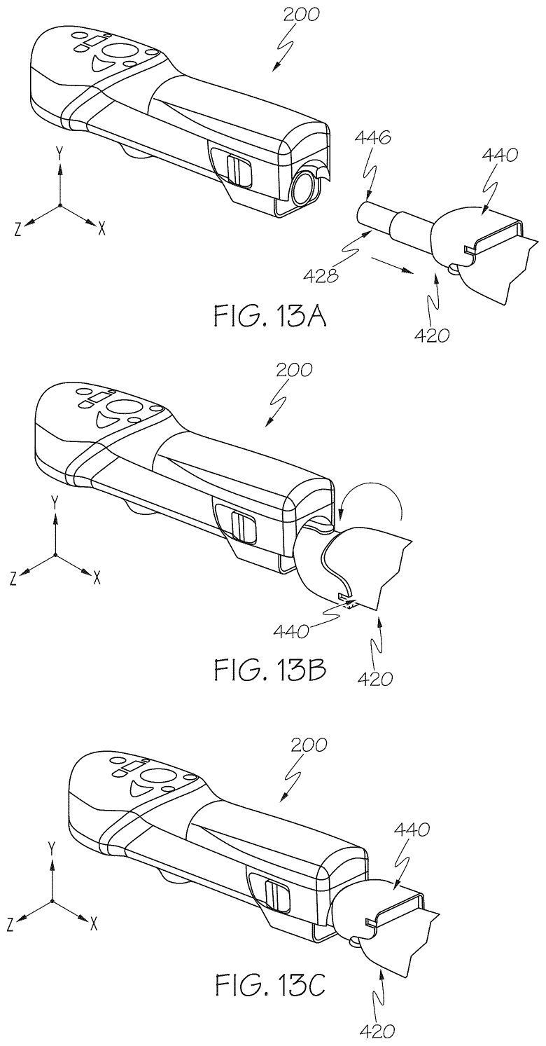

Turning now to FIGS. 13A-13C, installation of the marker delivery assembly 400 into the biopsy probe assembly 200 is depicted. Initially, as depicted in FIG. 10A, the shaft 428 of the integration module 420 may be inserted into the biopsy probe assembly 200. In these embodiments, the integration module 420 may be advanced in the longitudinal direction into the biopsy probe assembly 200 until the shaft 428 engages the inlet positioned in the biopsy probe assembly 200. In these embodiments, the shaft 428 may engage the inlet via a snap-fit, or other similar coupling, such that a user may hear and/or feel when the integration module has engaged the inlet of the biopsy probe assembly 200. It should be noted that, in these embodiments, the marker delivery assembly 400 may be preinstalled on the biopsy probe assembly 200 prior to performing a biopsy procedure, and may remain installed on the biopsy probe assembly 200 throughout the duration of the procedure.

Referring specifically to FIGS. 13B-13C, the integration module 420 may be rotated about a longitudinal axis in order to lock the adapter 446 (shown in FIG. 13A) to the inlet of the biopsy probe assembly 200. In these embodiments, the integration module 420 is inserted into the biopsy probe assembly 200 upside-down (e.g., such that the transmission cover 440 is facing downwardly, in the −y direction as depicted in the coordinate axis of FIGS. 13A-13C), as is depicted in FIG. 13B. Once the adapter 446 has engaged the inlet of the biopsy probe assembly 200, the integration module 420 is rotated about its longitudinal axis 180 degrees (e.g., such that the transmission cover 440 is facing upwardly, in the +y direction as depicted in the coordinate axis of FIGS. 13A-13C). As the integration module rotates, the adapter 446 may lock with the inlet of the biopsy probe assembly 200, thereby securing the marker delivery assembly 400 to the biopsy probe assembly 200.

In these embodiments, the marker delivery assembly 400 may be disengaged from the biopsy probe assembly 200 by rotating the integration module 420 in an opposite direction and withdrawing the marker delivery assembly 400 from the biopsy probe assembly. However, in some embodiments, the preinstalled marker delivery assembly 400 may be removed from the biopsy probe assembly 200 and replaced with a marker delivery assembly 400 having a different configuration.

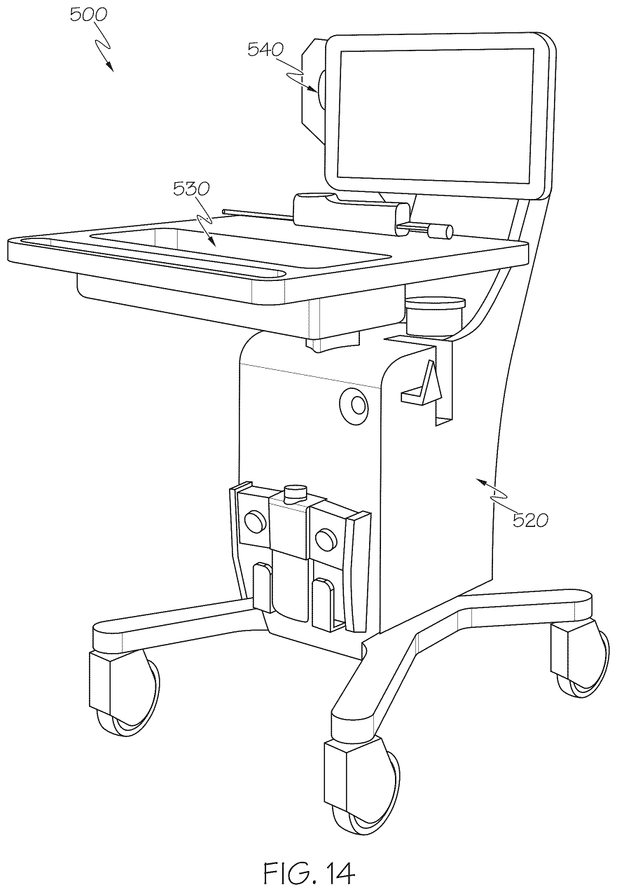

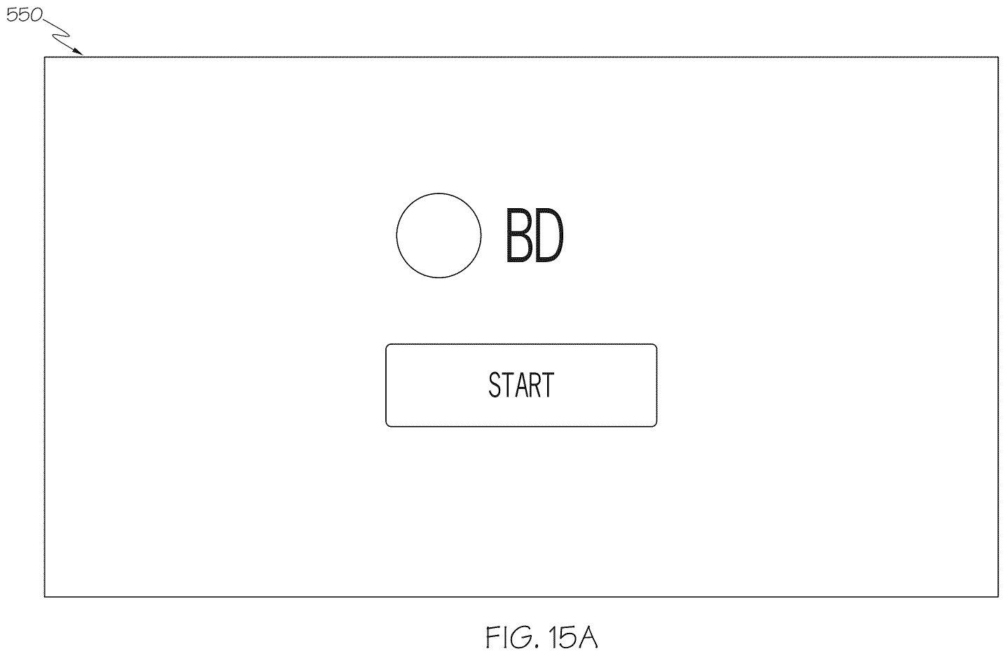

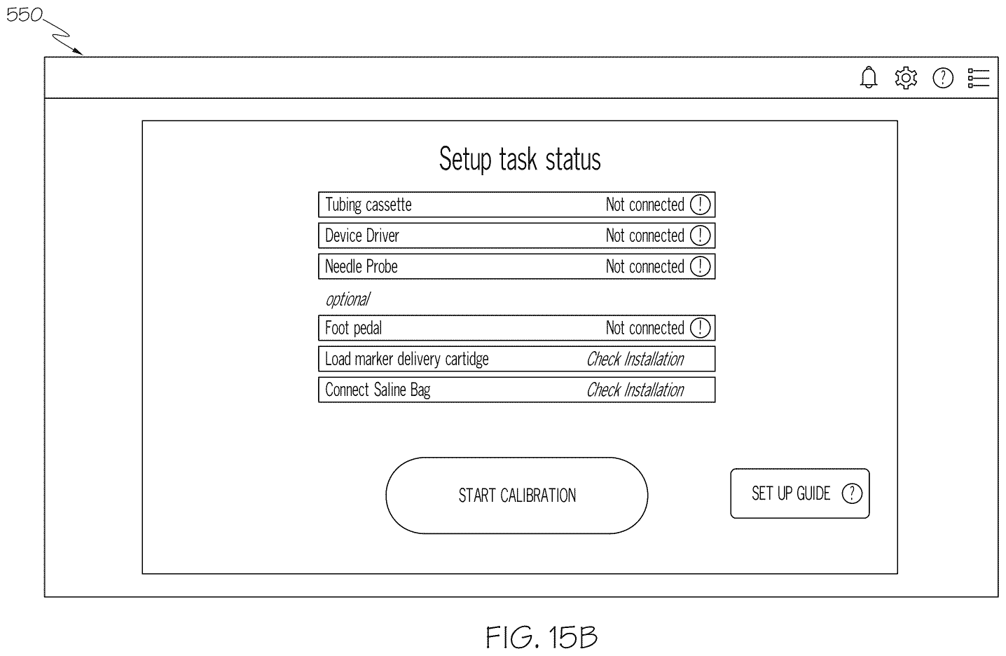

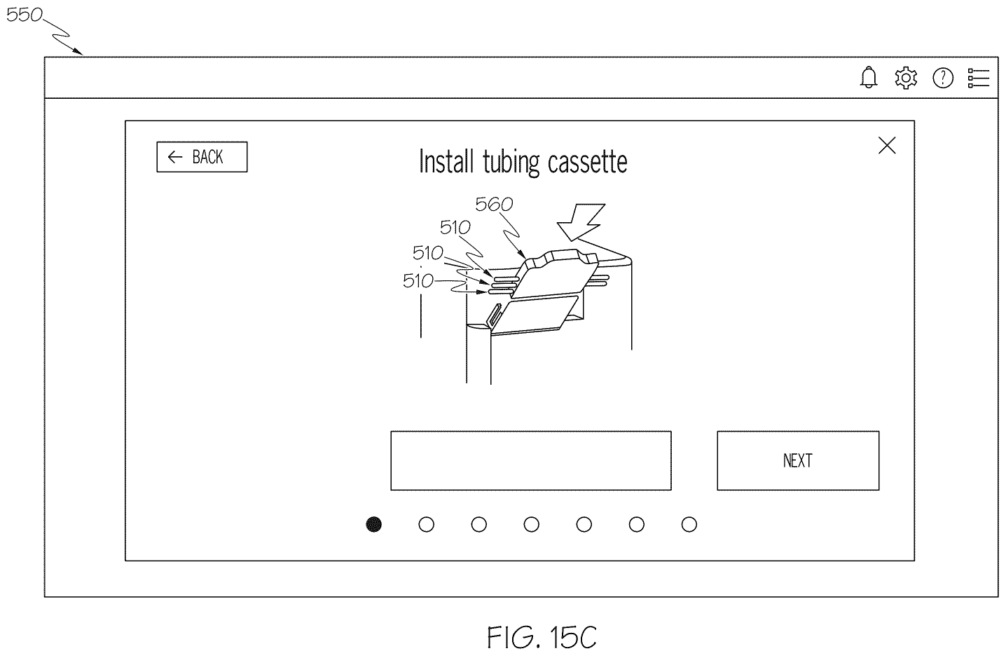

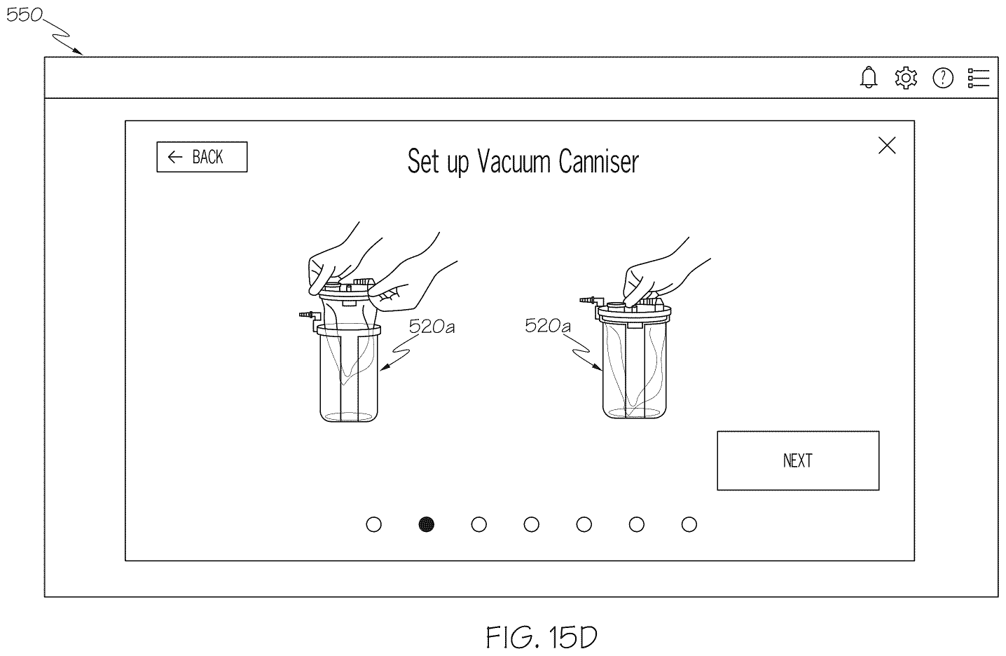

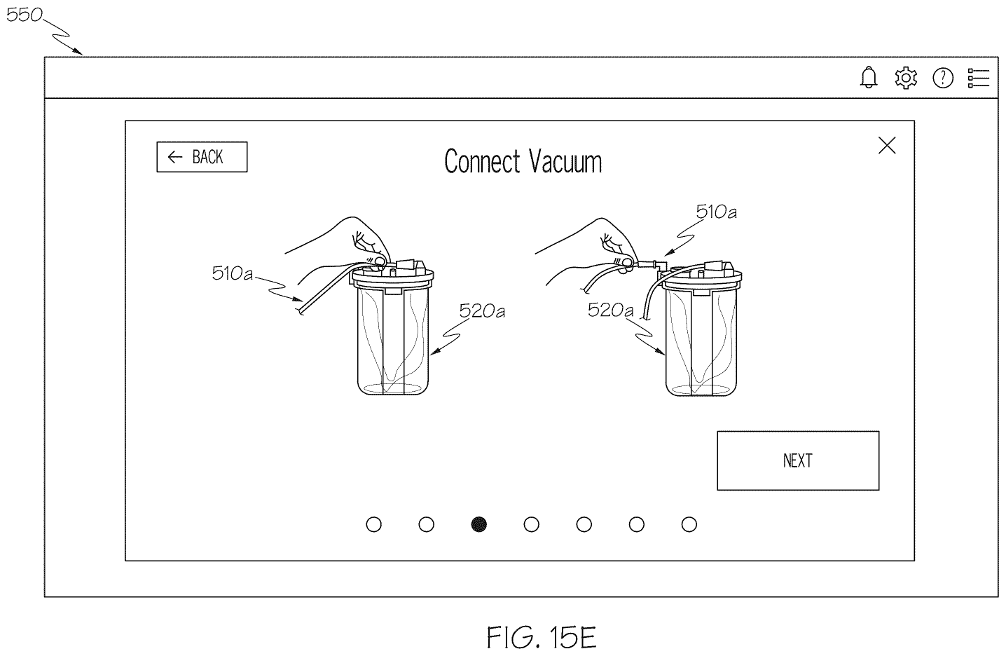

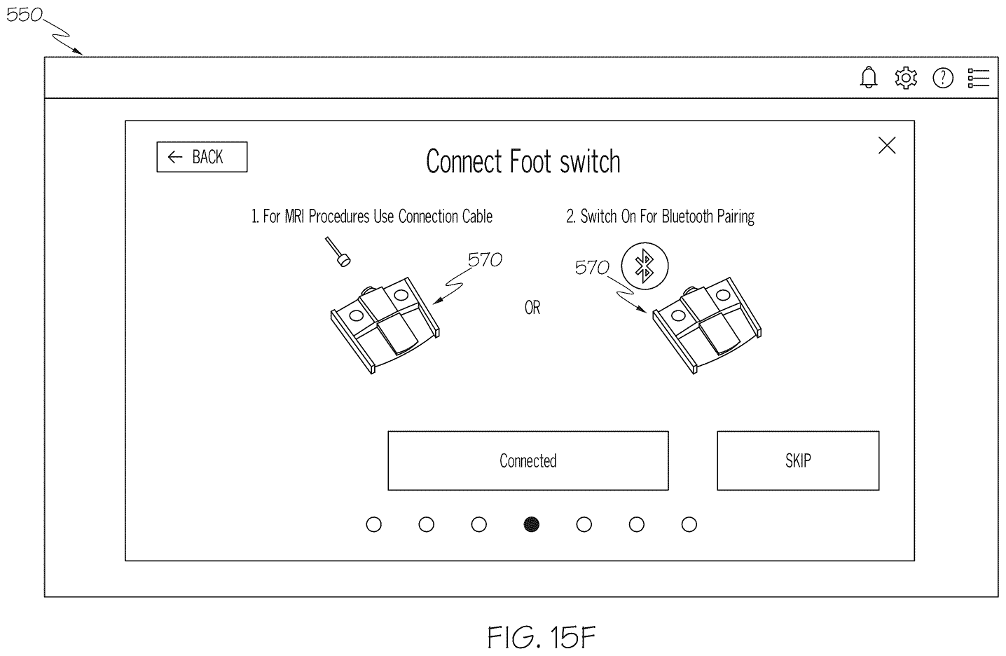

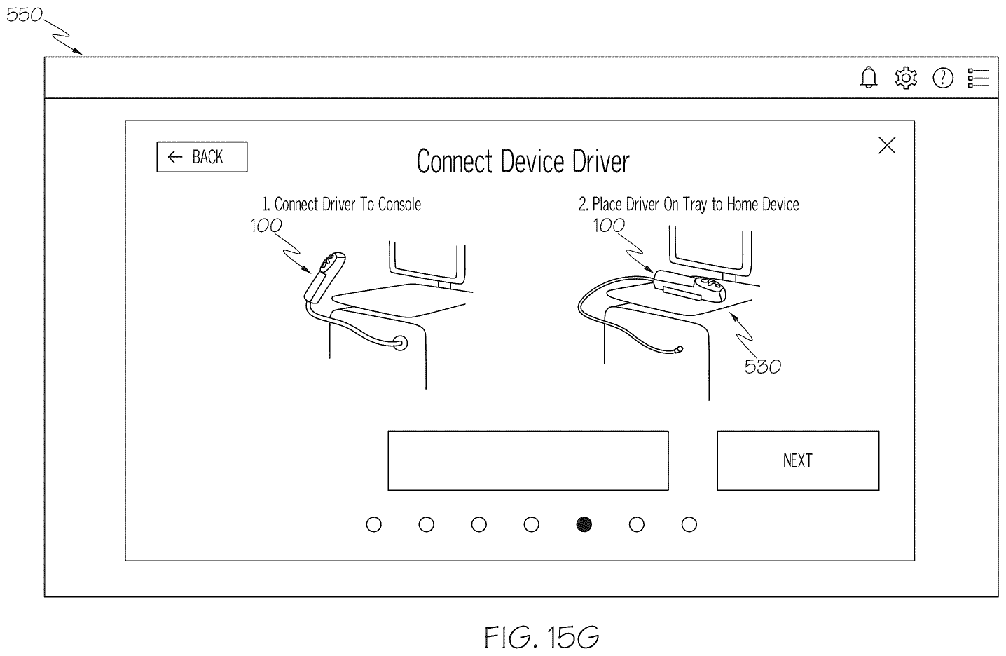

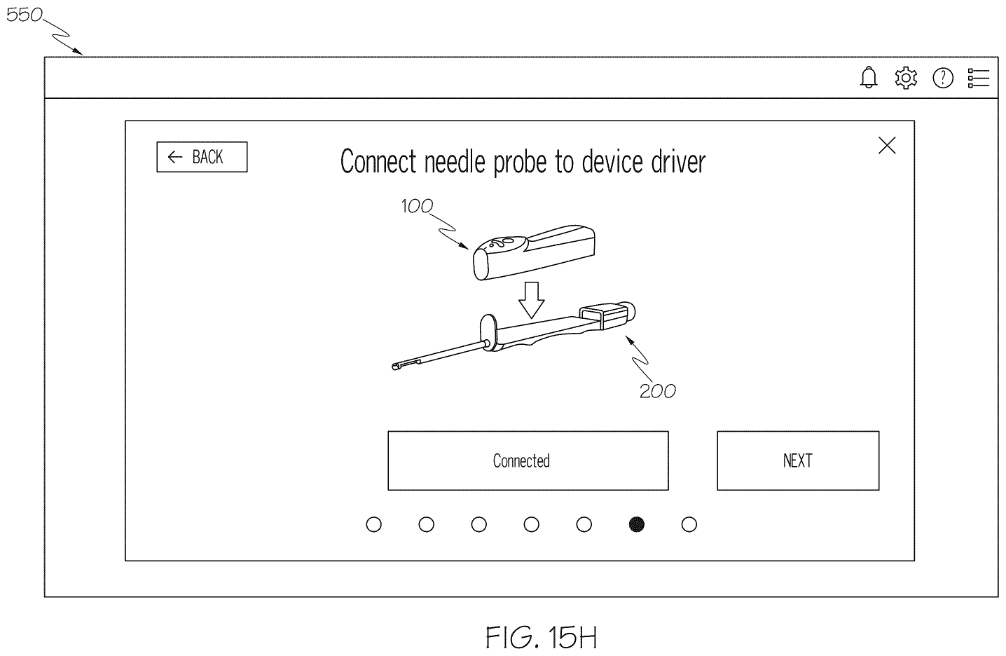

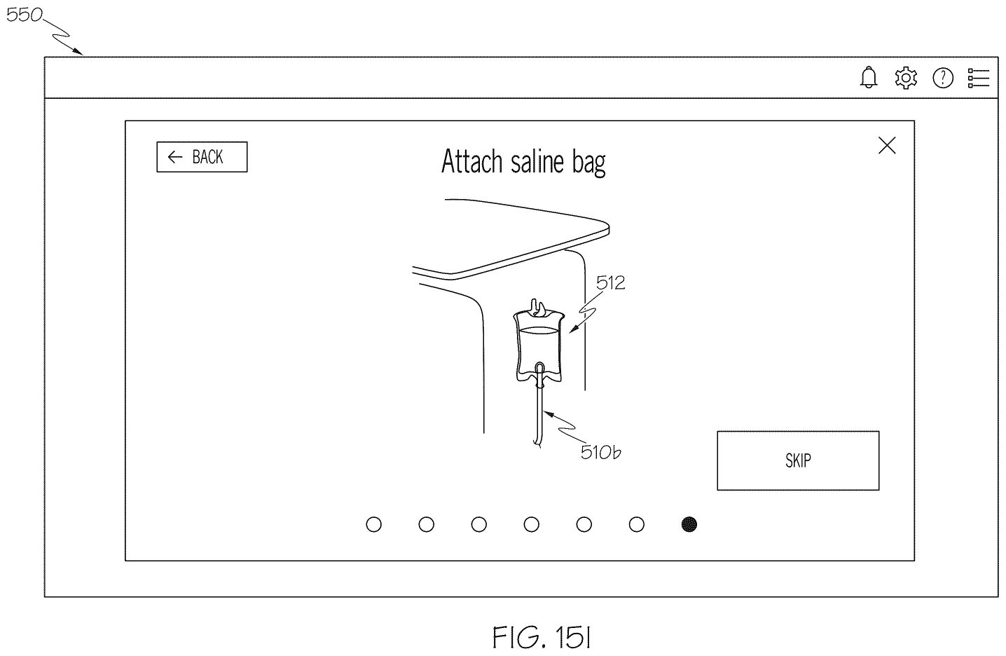

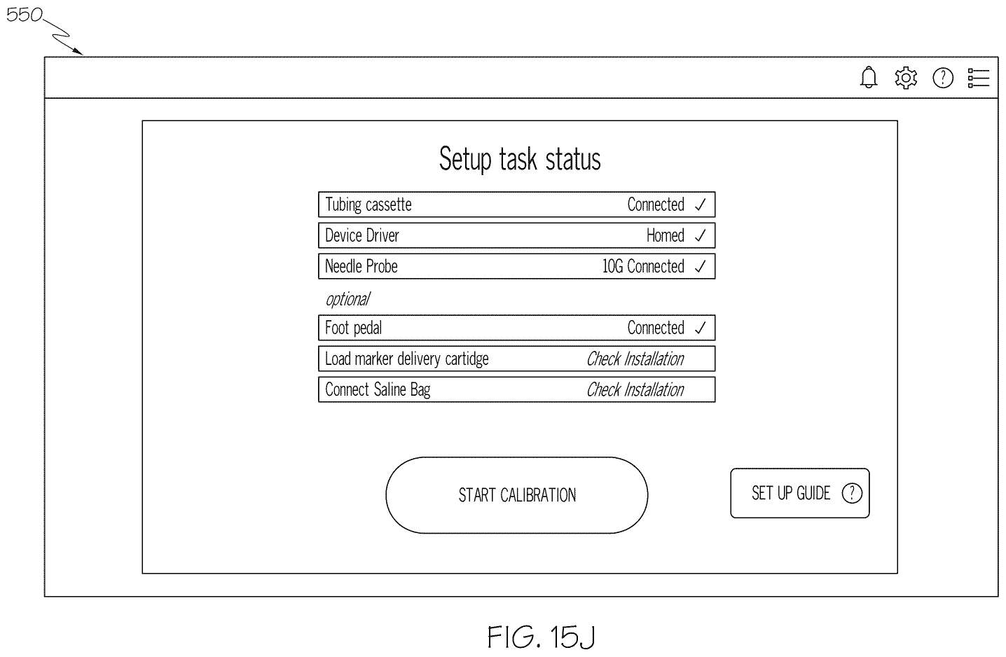

Turning now to FIGS. 14-15J, the biopsy device station 500 is generally illustrated. The biopsy device station 500, also referred to as a console, may include any number of computers, integrated circuits, chip assemblies, software, hardware, and the like for controlling one or more operations of the biopsy device. The biopsy device station 500 may comprise tubing 510, vacuum source 520, tray 530, and display 50. In some embodiments, the tubing 510 may comprise vacuum lines 510a, vent lines, saline lines 510b, and/or anesthetic lines. In some embodiments, other medication delivery lines may be included without departing from the scope of the present disclosure. In these embodiments, the vacuum line may be configured to connect the biopsy device 10 to the vacuum source 520 by way of vacuum canister 520a. The vacuum line 510a may engage vacuum fitting 348 of the outer basket housing 340, such that a vacuum may be applied by the vacuum canister 520a to biopsy device 10. In other embodiments, tubing 510 may comprise a saline line 510b connected to a saline bag 512 for providing a saline rinse through the biopsy device. In further embodiments still, tubing 510 may comprise an anesthetic line and may be configured to connect the biopsy device 10 to a syringe configured to provide anesthetic or other medication to the biopsy device 10. The tubing 510 may further be used as a venting line for the biopsy device 10, wherein the venting line is open to atmosphere.

In some embodiments, the biopsy device 10 may further include a cassette 560 having a plurality of solenoids (FIG. 15C). The cassette 560 may be configured around the tubing 510 such that the opening and closing of the plurality of solenoids may restrict the tubing 510. In these embodiments, the cassette 560 may be configured to control when vacuum, venting, salinc and/or anesthetics are applied to the biopsy device 10.

Turning now to FIGS. 15A-15J, the display 540 may include a graphic user interface (GUI) 550 which may be configured to guide a user through an initialization process of the biopsy device 10. As illustrated in FIG. 15A, the GUI may initially display a start screen comprising a start button, which may be designed to prompt a user to begin the initialization process. Once the initialization process is initiated, the GUI may be configured to display the status of a plurality of initialization tasks which may be completed prior to using the biopsy device 10. For example, as illustrated in FIG. 15B, the setup tasks may include required tasks and optional tasks. The required tasks may include installing the cassette 560, connecting the biopsy driver assembly 100 the biopsy device station 500 and connecting the biopsy probe assembly 200 to the biopsy driver assembly 100, or the like. Optional tasks may include connecting a foot pedal 570, loading a marker delivery mechanism and/or connecting saline bag 512. It is noted that these “required” and “optional” tasks are examples only, and any tasks may be considered options and/or required based on the particular implementation. When the required tasks are not completed, the tasks may be shown with a red indicator mark to illustrate the required tasks need attention. As further shown in FIG. 15B, a calibration button may be displayed on the GUI 550, which may not be activated at least until the required initialization tasks have been completed.

As illustrated in FIG. 15C, the GUI 550 may be configured to guide users through the initialization tasks. As shown in FIG. 15C, this may include instructing users how to install cassette 560, for example. Similarly, as shown in FIGS. 15D-15E, the GUI 550 may further guide the user through initialization of the vacuum canister 520a. Other initialization steps are contemplated and possible.

For example, in some embodiments, the GUI 550 may additionally walk a user through “optional” initialization tasks. As shown in FIG. 15F, the GUI 550 may further include a skip button for some tasks, for example, some tasks may be considered optional while other tasks may be considered mandatory, which may allow a user to bypass the optional initialization tasks and proceed with utilizing the biopsy device 10. The optional task illustrated in FIG. 15F may involve connecting a foot pedal 570 to the biopsy device station 500. In some embodiments, the foot pedal 570 may be connected to the biopsy device station 500 via a wired connection, while in other embodiments, the pedal 570 may be connected to the biopsy device station 500 wirelessly, such as via Bluetooth. In these embodiments, the GUI 550 may further comprise a connection indicator, which indicates when the pedal 570 has been successfully connected to the biopsy device station 500. Use of the pedal 570 may be desired, for example, when MRI procedures are being conducted using the biopsy device 10.

Turning now to FIG. 15G, the GUI 550 may instruct a user to connect the biopsy driver assembly 100 to the biopsy device station 500. The connection of the biopsy driver assembly 100 may include connecting the biopsy driver assembly 100 to the biopsy device station 500 using a wired connection, wireless connection, and/or mechanical tubing connectors (e.g., vacuum lines, fluid lines, etc.), and placing the connected biopsy driver assembly 100 onto the tray 530 to home the biopsy driver assembly 100. As illustrated in FIG. 15G, connecting the biopsy driver assembly 100 to the biopsy device station 500 may be considered a required task for initialization purposes.

With the biopsy driver assembly 100 connected, the GUI 550 may instruct a user to connect the biopsy probe assembly 200 to the biopsy driver assembly 100. The process of attaching the biopsy driver assembly 100 to the biopsy probe assembly 200 has been described with reference to FIGS. 5 and 6. The GUI 550 may then instruct the user to attach saline bag 512 to the biopsy device 10 as an optional task (FIG. 15I), for example.

Once each of the initialization tasks have been completed and/skipped depending on the mandatory/optional status of each task, the GUI 550 may again provide the user with the status of each of the tasks, as was previously illustrated in FIG. 15B. In this embodiment, tasks which have been successfully completed may be marked with a check, such as a green check.

When each of the necessary tasks have been successfully completed, the GUI 550 may prompt a user to calibrate the biopsy device 10 prior to conducting a biopsy. For example, calibration may include a calibration routine wherein different drives of the biopsy device are operated to ensure appropriate operation. For example, the biopsy device may include any number of sensors to determine positions of one or more components of the biopsy device to determine proper operation. In some embodiments, calibration procedures may include priming fluid pathways with fluid, testing vacuum, or the like.

The calibration process may utilize one or more sensors such as a plurality of sensors, such as infrared sensors, rotation sensors, current sensors, power sensors, positional sensors, or the like, to determine that the biopsy device 10 is prepared for biopsy operations. For example, the calibration process may involve operating motors and monitoring power draw to determine the position of the cutting gear spindle and/or the piercing module (e.g., via hard-stop homing). Other steps of the calibration process may include using one or more sensors (e.g., the IR sensor) to detect the proper components are installed or otherwise properly position on or within the biopsy device (e.g., the biopsy probe assembly 200, sample basket assembly 300, the piercing module, the cutting gear spindle, etc.) prior to performing a biopsy procedure, such that a user may confirm that the necessary components have been attached to the biopsy driver assembly 100 prior to performing the biopsy procedure. The one or more sensors (such as an IR sensors) may be used to determine the type of biopsy probe assembly 200 attached to the biopsy driver assembly 100, such that a user may confirm that the appropriate biopsy probe assembly 200 is attached prior to performing the biopsy procedure. For example, the biopsy probe assembly 200 may include a unique identifier (e.g., bar code, chip, etc.) which allows for identification of the biopsy probe assembly 200 by the system. Such identification procedure may occur during operation of a motor to rotate or otherwise move the target identifier for full scanning. In some embodiments, the calibration process may further involve confirming operation of the vacuum source, which may include monitoring a pressure transducer in communication with the vacuum canister (console based) to confirm target pressure is reached and ensure no significant leaks are present, In some embodiments, the pressure transducer may be within the biopsy device. Other calibration procedures may include confirming the position of the piercing module 126 and/or determining if a marker has been installed within the biopsy probe assembly 200.

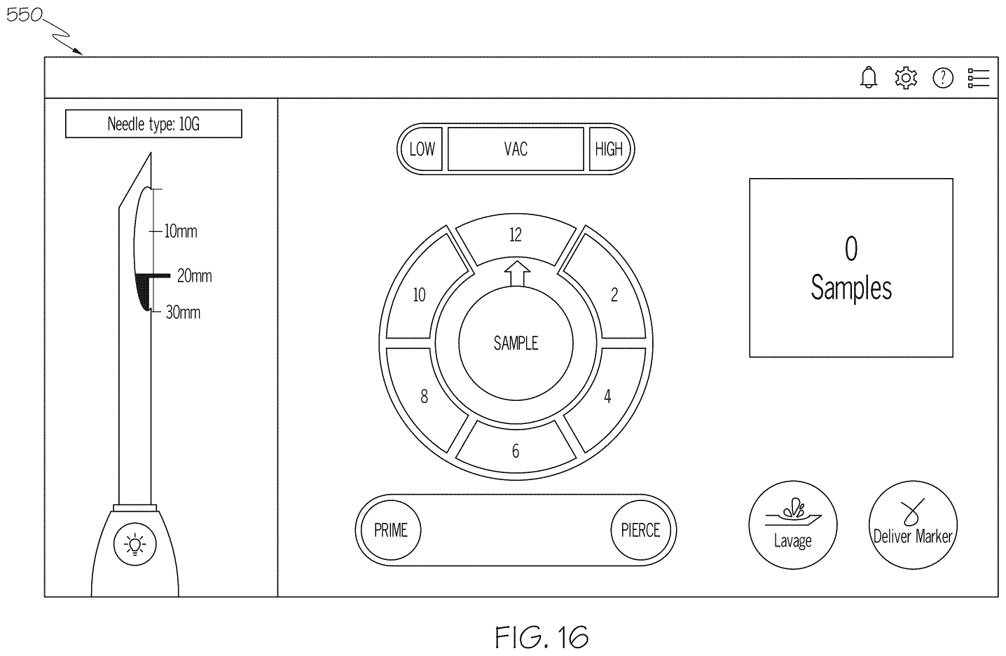

After the biopsy device station 500 has calibrated the biopsy device 10, the biopsy device may be used to conduct a biopsy, e.g., by inserting and/or firing the sample notch cannula into the tissue at a desired biopsy site, and taking samples as described herein. While the biopsy device 10 is operating, the GUI 550 may display a plurality of visual indicators which indicate the status and/or position of one or more components of the biopsy device 10. As shown in FIG. 16, the display may indicate the rotational position of the sample notch cannula 220, the axial position of the cutter cannula 230, the amount of vacuum being supplied by the vacuum source 520, the status of the piercing module 126, and/or the number of tissue samples collected by the biopsy device 10. In operation, the GUI 550 may assist a user in documenting the use of the biopsy device, which may ensure that the desired size and number of tissue samples are obtained during a single biopsy procedure.

In some embodiments, the GUI 550 may provide a user interface allowing the user to control the operation of the biopsy device 10. For example, the GUI 550 may be a touchscreen operable to receive user inputs thereon. In other embodiments, user interface devices may be included such as a keyboard, mouse, buttons, toggles, switches, or the like. In these embodiments, a user may interact with the GUI to control various components within the biopsy device 10 rather than or in addition to utilizing controller 130. For example, the GUI 550 may be configured to cause the biopsy device 10 to obtain a sample when the “sample” button, as illustrated in FIG. 16, is activated. The GUI 550 shown in FIG. 16 may be similarly configured to control the longitudinal position of the cutter cannula 230, the rotational position of the sample notch cannula 220, the position of the piercing module 126 and the amount of vacuum supplied by the vacuum source 520.

A tissue sample severing and transporting sequence using the biopsy device 10 will now be described in detail herein. Initially, tubing 510, including vacuum line, venting line, saline line and/or anesthetic line, may already be installed into the cassette 560. With the cassette 560 configured, the tubing 510 may then be attached to the biopsy probe assembly 200, such that the vacuum, vacuum level, venting, saline, and/or anesthetic delivery may be controlled. Specifically, as the plurality of solenoids of the cassette 560 move between a closed position and an open position, the cassette 560 may restrict the ability of fluid to pass through tubing 510. For instance, when the plurality of solenoids are in the closed position, the cassette 560 may restrict the tubing 510 such that air, saline, anesthetic, or other fluid cannot flow through the tubing 510. In contrast, when the plurality of solenoids are moved to the open position, fluid may pass through the tubing 510 and into the biopsy device 10.

Once the tubing 510 and cassette 560 are loaded, the biopsy driver assembly 100 may be coupled to the biopsy probe assembly 200. For example, the biopsy driver assembly 100 may be coupled to the biopsy probe assembly 200 by aligning the retaining hooks 212a of the probe housing 210 with the slots 110a of the driver housing 110 and sliding the driver housing 110 in a distal direction to lock the biopsy driver assembly 100 and biopsy probe assembly 200 together.

With the biopsy device 10 assembled and coupled to vacuum source 520, the biopsy device is inserted or fired (as described above) into a target site, with the piercing tip 226 of the sample notch cannula 220 piercing tissue until the biopsy device 10 is positioned at the target site. Once the biopsy device has been appropriately positioned, the vacuum source 520 is activated and the vacuum line is opened. As vacuum is applied to the biopsy device 10, the cutter module 122 is activated, such that the drive gear 122b of the cutter module 122 drives the cutter gear-spindle set 232 backwards. Specifically, drive gear 122b may rotate in a clockwise direction, which in turn may drive driven gear 232a in the opposite, counterclockwise direction. As the driven gear 232a rotates in the counterclockwise direction, the spindle 232b may move in a proximal direction such that the cutter gear-spindle set 232 and cutter cannula 230 moves in a proximal direction. As the cutter gear-spindle set 232 is driven backwards, the cutter cannula 230 retracts within the sample notch cannula 220, such that the sample notch 224 of the sample notch cannula 220 is opened. It is noted that the size to which the sample notch is opened may be controlled. For example, the sample opening of the sample notch may be adjusted via positioning of the cutter cannula 230. With the sample notch 224 exposed, the vacuum applied to the biopsy device 10 draws tissue into the sample notch 224. Once the tissue has been drawn into the sample notch 224, the cutter module 122 drives drive gear 122b in the reverse direction, such that the cutter gear-spindle set 232 is driven forward or distally, thereby moving the cutter cannula 230 in the distal direction towards the sample notch 224. As the cutter cannula 230 moves across the sample notch 224, tissue is excised from the target site. The cassette may then open the vacuum line 510a, which allows airflow to pass into the fluid pathway 250 between the sample notch cannula 220 and cutter cannula 230. As airflow travels into the fluid pathway 250, the vacuum may pull the excised tissue through the cutter cannula 230 in a proximal direction such that the excised tissue traverses the length of the cutter cannula 230 and is deposited in the sample basket assembly 300, further described above. The sample basket assembly 300 may store excised tissue transported through the cutter cannula 230 until the desired number of tissue samples have been excised. Once the desired number of tissue samples are obtained, the vacuum source 520 may be deactivated and the tissue samples may be collected from the sample basket assembly 300.

The biopsy device 10 may be further configured to provide a saline rinse of the tissue samples collected in the sample basket assembly 300. In these embodiments, a bag of saline may be connected to the saline line. With the saline connected, the biopsy device station 500 may be configured to control the cassette 560 to open the vacuum line while leaving the vent line closed. The vacuum may then pull saline from the saline bag through the saline line and into the biopsy probe assembly 200. The saline may flow through the fluid pathway 250 between the sample notch cannula 220 and cutter cannula 230. As the saline flows through the fluid pathway 250, the vacuum may draw the saline through the cutter cannula 230 and into the sample basket assembly 300. As the saline is drawn through the cutter cannula 230 and into the sample basket assembly 300, excess blood may be washed away from the hinge basket assembly 360 of the sample basket assembly 300, which may enable a user to confirm that tissue samples have been delivered to the sample basket assembly 300. Additional lines for fluid waste may be included to remove fluid waste from the sample basket assembly 300. in some embodiments, it is contemplated that the saline line may be configured to enter the sample basket assembly 300, such that the vacuum may pull saline from the saline bag through the saline line and directly into the sample basket assembly 300 to rinse collected tissue samples. In these embodiments, additional lines may still be utilized to remove fluid waste from the sample basket assembly 300.

In some embodiments, the biopsy device 10 may be configured to provide anesthetics to a target site. In these embodiments, a syringe containing anesthetics may be connected to the saline line. With the syringe connected, the vacuum source 520 may be turned off and each of the plurality of solenoids of the cassette 560 may be moved to the closed position such that all of the tubing 510 is closed. With tubing 510 closed, the syringe may push anesthetics into the biopsy device 10 such that the anesthetics fill the fluid pathway 250 between the cutter cannula 230 and the sample notch cannula 220. As the anesthetics traverse the fluid pathway 250 in the distal direction, the anesthetics may be discharged into the target site by way of the sample notch 224. In these embodiments, the biopsy device 10 may include additional control valves configured to prevent the backflow of fluid away from the biopsy probe assembly 200.

As has been described herein, in some embodiments, the tissue sample severing and transporting sequence may further include delivery of a marker to a target site using the marker delivery assembly 400. In these embodiments, the marker delivery assembly 400 may be coupled to the biopsy probe assembly 200 via the integration module 420, as has been described herein. More particularly, the integration module 420 may be inserted into the biopsy probe assembly 200 until the adapter 446 engages the inlet 238 of the biopsy probe assembly 200 (FIGS. 7A-7B, 9A-12C).

Once the marker delivery assembly 400 is properly secured to the biopsy probe assembly 200, the cutter cannula 230 may be withdrawn to the fully retracted position, such that the spindle 232b disengages the spindle nut 232c and enters free rotation zone 232d. With the cutter cannula 230 in the fully retracted position, the spindle 232b may engage the spindle gear 430 of the integration module 420, such that rotation of the spindle 232b may drive the spindle gear 430.

As the spindle gear 430 rotates, the shaft 428 and drum gear 432 may similarly rotate. In these embodiments, rotation of the drum gear 432 may result in rotation of the bevel drum 434. As has been described herein, the bevel drum 434 may be in frictional engagement with the pushrod 470, such that rotation of the bevel drum 434 cause the pushrod 470 to unwind.

As the pushrod 470 unwinds, the pushrod 470 may extend into and through the chamber 451 of the marker delivery cartridge 452. In these embodiments, as the pushrod 470 traverses the chamber 451, the pushrod 470 may engage at least one of a plurality of markers 480 positioned within the chamber 451. As the pushrod 470 continues to advance, the pushrod 470 may force the at least one of the plurality of markers 80 out of the chamber 451 and into the cutter cannula 230. In these embodiments, the pushrod 470 may continue to advance through the cutter cannula 230 until the marker 480 is forced out of the sample notch 224 and into the target site (FIG. 12C).

In view of the foregoing, it should be understood that a biopsy device is disclosed herein. The biopsy device may include marker delivery assembly having an integration module and a marker delivery module. The integration module may include a connector having a cavity and a neck that extends outwardly from the cavity in a longitudinal direction. A transmission cover having a shaft is coaxially disposed about the neck of the connector, the shaft further extending between a distal end having a spindle gear and a proximal end having a drum gear. A bevel drum is rotatably coupled to the drum gear, and a pushrod frictionally engaged to the bevel drum. The marker delivery module may include a marker delivery housing that engages the cavity of the connector to releasably couple the marker delivery module to the integration module, and may further include at least one coupling rod and at least one fulcrum rod. The marker delivery module may further include a marker delivery cartridge rotatable between an initial position in which the marker delivery cartridge engages the at least one coupling rod, and a delivery position in which the marker delivery cartridge is rotated about the at least one fulcrum rod. The marker delivery cartridge may further include a chamber that houses a plurality of markers. Rotation of the bevel drum may cause the pushrod to unwind, such that the pushrod extends through the chamber of the marker delivery cartridge and forces at least one of the plurality of markers into the biopsy device.

Embodiments may be further described with respect to the following numbered clauses:

Clause 1. A marker delivery assembly for a biopsy device comprising: an integration module comprising: a connector defining a cavity and comprising a neck that extends outwardly from the cavity in a longitudinal direction; a transmission cover having a shaft coaxially disposed about the neck of the connector, the shaft further extending between a distal end having a spindle gear and a proximal end having a drum gear; a bevel drum rotatably coupled to the drum gear; and a pushrod frictionally engaged to the bevel drum; and a marker delivery module comprising: a marker delivery housing that fits within the cavity of the connector to releasably couple the marker delivery module to the integration module, the marker delivery housing having at least one coupling rod and at least one fulcrum rod; and a marker delivery cartridge rotatable between an initial position in which the marker delivery cartridge engages the at least one coupling rod, and a delivery position in which the marker delivery cartridge is rotated about the at least one fulcrum rod, the marker delivery cartridge further having a chamber that houses a plurality of markers; wherein, when the marker delivery cartridge is in the delivery position, rotation of the bevel drum causes the pushrod to unwind, such that the pushrod extends through the chamber of the marker delivery cartridge and forces at least one of the plurality of markers into the biopsy device.

Clause 2. The marker delivery assembly of clause 1, wherein the marker delivery module further defines at least one slot and the marker delivery cartridge comprises at least one latch.

Clause 3. The marker delivery assembly of clause 1 or 2, wherein the at least one latch engages the at least one slot when the marker delivery cartridge is in the delivery position.

Clause 4. The marker delivery assembly of any of clauses 1-3, wherein the transmission cover further includes an adapter for releasably coupling the marker delivery assembly to the biopsy device.

Clause 5. The marker delivery assembly of any of clauses 1-4, wherein the shaft is rotatably disposed about the neck of the connector, such that rotation of the spindle gear causes rotation of the shaft and the drum gear.

Clause 6. The marker delivery assembly of any of clauses 1-5, wherein the plurality of markers are preloaded into the chamber of the marker delivery cartridge.

Clause 7. The marker delivery assembly of any of clauses 1-6, wherein when the marker delivery housing engages the connector, the transmission cover contacts the marker delivery cartridge.

Clause 8. The marker delivery assembly of any of clauses 1-7, wherein contact between the transmission cover and the marker delivery cartridge rotates the marker delivery cartridge from the initial position to the delivery position.

Clause 9. The marker delivery assembly of any of clauses 1-8, wherein the marker delivery cartridge further comprises a ramp positioned in the chamber.

Clause 10. The marker delivery assembly of any of clauses 1-9, wherein, when the marker delivery cartridge is in the delivery position, rotation of the bevel drum causes the pushrod to unwind, such that the pushrod extends through the chamber of the marker delivery cartridge and forces at least one of the plurality of markers and the ramp into the biopsy device.

Clause 11. The marker delivery assembly of any of clauses 1-10, wherein, when the marker delivery cartridge is in the initial position, the pushrod is prevented from traversing the chamber of the marker delivery cartridge.

Clause 12. A biopsy device, comprising: a driver assembly; a biopsy probe assembly releasably attached to the driver assembly, the biopsy probe assembly having a cutter cannula and a sample notch cannula coaxially arranged along a longitudinal axis, the cutter cannula being positioned inside the sample notch cannula to define a fluid pathway between the cutter cannula and the sample notch cannula; a marker delivery assembly comprising: an integration module comprising: a connector having a cavity and a neck that extends outwardly from the cavity in a longitudinal direction, the neck being insertable into the biopsy probe assembly; a transmission cover having a shaft coaxially disposed about the neck of the connector, the shaft further extending between a distal end having a spindle gear and a proximal end having a drum gear; a bevel drum rotatably coupled to the drum gear; and a pushrod frictionally engaged to the bevel drum; and a marker delivery module comprising: a marker delivery housing that engages the cavity of the connector to releasably couple the marker delivery module to the integration module, the marker delivery housing having at least one coupling rod and at least one fulcrum rod; and a marker delivery cartridge rotatable between an initial position in which the marker delivery cartridge engages the at least one coupling rod, and a delivery position in which the marker delivery cartridge is rotated about the at least one fulcrum rod, the marker delivery cartridge further having a chamber that houses a plurality of markers; and a sample basket assembly releasably attached to the marker delivery module of the marker delivery assembly, the sample basket assembly having a vacuum port for connecting the fluid pathway in fluid communication with a vacuum source, a venting line for connecting the fluid pathway to atmosphere, and a saline port for connecting the fluid pathway to a saline source.

Clause 13. The biopsy device of clause 12, wherein the cutter cannula further includes a spindle and a driven gear for translating the cutter cannula between an extended position and a fully retracted position.

Clause 14. The biopsy device of clause 12 or 13, wherein the spindle engages the driven gear when the cutter cannula is in the extended position, and the spindle engages the spindle gear of the integration module when the cutter cannula is in the fully retracted position.

Clause 15. The biopsy device of any of clauses 12-14, wherein, when the cutter cannula is in the fully retracted position and the marker delivery cartridge is in the delivery position, rotation of the bevel drum causes the pushrod to unwind, such that the pushrod extends through the chamber of the marker delivery cartridge and forces at least one of the plurality of markers through the cutter cannula and out of the sample notch cannula.

Clause 16. The biopsy device of any of clauses 12-15, wherein the biopsy probe assembly further includes an inlet and the transmission cover further includes an adapter, the adapter being configured to engage the inlet and couple the marker delivery assembly to the biopsy probe assembly.

Clause 17. The biopsy device of any of clauses 12-16, wherein the marker delivery cartridge further comprises a ramp positioned in the chamber.

Clause 18. The biopsy device of any of clauses 12-17, wherein, when the cutter cannula is in the fully retracted position and the marker delivery cartridge is in the delivery position, rotation of the bevel drum causes the pushrod to unwind, such that the pushrod extends through the chamber of the marker delivery cartridge and forces at least one of the plurality of markers and the ramp through the cutter cannula.

Clause 19. A method of using a biopsy device, the method comprising: releasably coupling a marker delivery assembly to the biopsy device, the marker delivery assembly including a bevel drum coupled to a pushrod, and a marker delivery cartridge rotatable between an initial position and a delivery position; rotating the marker delivery cartridge from the initial position to the delivery position; rotating the bevel drum, such that the rotation of the bevel drum extends the pushrod into and through the marker delivery cartridge; contacting, with the pushrod, at least one of a plurality of markers housed within the marker delivery cartridge; pushing, with the pushrod, the least one of the plurality of markers housed within the marker delivery cartridge out of the marker delivery cartridge and into the biopsy device; and deploying, using the pushrod, the at least one of the plurality of markers from the biopsy device.

Clause 20. The method of clause 19, further comprising repositioning the biopsy device at a second target site and deploying another of the at least one of the plurality of markers.

Clause 21. A marker delivery assembly for a biopsy device comprising: an integration module comprising: a connector; a transmission cover having a shaft; and a pushrod rotatably coupled to the shaft; and a marker delivery module comprising: a marker delivery housing that is releasably coupled to the connector of the integration module; and a marker delivery cartridge including a chamber that houses at least one marker; wherein the marker delivery cartridge is rotatably coupled to the marker delivery housing.

Clause 22. The marker delivery assembly of clause 21, wherein the marker delivery cartridge is rotatable between an initial position and a delivery position.

Clause 23. The marker delivery assembly of clause 21 or 22, wherein the marker delivery cartridge deploys the at least one marker in the delivery position.

Clause 24. The marker delivery assembly of any of clauses 21-23, wherein rotation of the shaft of the integration module causes the pushrod to extend through the chamber of the marker delivery housing.

Clause 25. The marker delivery assembly of any of clauses 21-24, wherein the marker delivery housing further includes at least one coupling shaft that rotatably couples the marker delivery cartridge to the marker delivery shaft.

Clause 26. The marker delivery assembly of any of clauses 21-25, wherein the marker delivery housing further includes at least one fulcrum shaft, and the marker delivery cartridge rotates about the at least one fulcrum shaft.

Clause 27. The marker delivery assembly of any of clauses 21-26, wherein the pushrod is a nitinol wire.

Clause 28. A biopsy device comprising: a driver assembly; a biopsy probe assembly releasably attached to the driver assembly; a marker delivery assembly releasably attached to the biopsy probe assembly, such that the marker delivery assembly is in fluid communication with the biopsy probe assembly; and a sample basket assembly releasably attached to the marker delivery assembly, such that the sample basket assembly is in fluid communication with the marker delivery assembly and the biopsy probe assembly.

Clause 29. The biopsy device of clause 28, wherein the marker delivery assembly comprises an integration module that releasably attaches the marker delivery assembly to the biopsy probe assembly and a marker delivery module that releasably attaches the marker delivery assembly to the sample basket assembly.

Clause 30. The biopsy device of clause 29, wherein the marker delivery module of the marker delivery assembly further includes a marker, the marker being deployable from the marker delivery assembly to the biopsy probe assembly.

As used herein, “substantially”, “slightly”, “approximately” and other words of degree are relative modifiers intended to indicate permissible variation from the characteristic so modified. It is not intended to be limited to the absolute value or characteristic which it modifies but rather possessing more of the physical or functional characteristic than its opposite, and approaching or approximating such a physical or functional characteristic.

Also, as used herein, the term “coupled”, and its derivatives, is intended to embrace any operationally functional connection, i.e., a direct connection or an indirect connection.

While the present disclosure has been described with respect to at least one embodiment, the present disclosure can be further modified within the spirit and scope of this disclosure. This application is therefore intended to cover any variations, uses, or adaptations of the present disclosure using its general principles.

Additional embodiments, supplements, and aspects of the present disclosure are provided in the Appendix.

1. A marker delivery assembly for a biopsy device comprising:
an integration module comprising:
a connector defining a cavity and comprising a neck that extends outwardly from the cavity in a longitudinal direction;
a transmission cover having a shaft coaxially disposed about the neck of the connector, the shaft further extending between a distal end having a spindle gear and a proximal end having a drum gear;
a bevel drum rotatably coupled to the drum gear; and
a pushrod frictionally engaged to the bevel drum; and
a marker delivery module comprising:
a marker delivery housing that fits within the cavity of the connector to releasably couple the marker delivery module to the integration module, the marker delivery housing having at least one coupling rod and at least one fulcrum rod; and
a marker delivery cartridge rotatable between an initial position in which the marker delivery cartridge engages the at least one coupling rod, and a delivery position in which the marker delivery cartridge is rotated about the at least one fulcrum rod, the marker delivery cartridge further having a chamber that houses a plurality of markers;
wherein, when the marker delivery cartridge is in the delivery position, rotation of the bevel drum causes the pushrod to unwind, such that the pushrod extends through the chamber of the marker delivery cartridge and forces at least one of the plurality of markers into the biopsy device.

2. The marker delivery assembly of claim 1, wherein the marker delivery module further defines at least one slot and the marker delivery cartridge comprises at least one latch.

3. The marker delivery assembly of claim 2, wherein the at least one latch engages the at least one slot when the marker delivery cartridge is in the delivery position.

4. The marker delivery assembly of claim 1, wherein the transmission cover further includes an adapter for releasably coupling the marker delivery assembly to the biopsy device.

5. The marker delivery assembly of claim 1, wherein the shaft is rotatably disposed about the neck of the connector, such that rotation of the spindle gear causes rotation of the shaft and the drum gear.

6. The marker delivery assembly of claim 1, wherein the plurality of markers are preloaded into the chamber of the marker delivery cartridge.

7. The marker delivery assembly of claim 1, wherein when the marker delivery housing engages the connector, the transmission cover contacts the marker delivery cartridge.

8. The marker delivery assembly of claim 7, wherein contact between the transmission cover and the marker delivery cartridge rotates the marker delivery cartridge from the initial position to the delivery position.

9. The marker delivery assembly of claim 1, wherein the marker delivery cartridge further comprises a ramp positioned in the chamber.

10. The marker delivery assembly of claim 9, wherein, when the marker delivery cartridge is in the delivery position, rotation of the bevel drum causes the pushrod to unwind, such that the pushrod extends through the chamber of the marker delivery cartridge and forces at least one of the plurality of markers and the ramp into the biopsy device.

11. The marker delivery assembly of claim 1, wherein, when the marker delivery cartridge is in the initial position, the pushrod is prevented from traversing the chamber of the marker delivery cartridge.

12. A biopsy device, comprising:
a driver assembly;
a biopsy probe assembly releasably attached to the driver assembly, the biopsy probe assembly having a cutter cannula and a sample notch cannula coaxially arranged along a longitudinal axis, the cutter cannula being positioned inside the sample notch cannula to define a fluid pathway between the cutter cannula and the sample notch cannula;
a marker delivery assembly comprising:
an integration module comprising:
a connector having a cavity and a neck that extends outwardly from the cavity in a longitudinal direction, the neck being insertable into the biopsy probe assembly;
a transmission cover having a shaft coaxially disposed about the neck of the connector, the shaft further extending between a distal end having a spindle gear and a proximal end having a drum gear;
a bevel drum rotatably coupled to the drum gear; and
a pushrod frictionally engaged to the bevel drum; and
a marker delivery module comprising:
a marker delivery housing that engages the cavity of the connector to releasably couple the marker delivery module to the integration module, the marker delivery housing having at least one coupling rod and at least one fulcrum rod; and
a marker delivery cartridge rotatable between an initial position in which the marker delivery cartridge engages the at least one coupling rod, and a delivery position in which the marker delivery cartridge is rotated about the at least one fulcrum rod, the marker delivery cartridge further having a chamber that houses a plurality of markers; and
a sample basket assembly releasably attached to the marker delivery module of the marker delivery assembly, the sample basket assembly having a vacuum port for connecting the fluid pathway in fluid communication with a vacuum source, a venting line for connecting the fluid pathway to atmosphere, and a saline port for connecting the fluid pathway to a saline source.

13. The biopsy device of claim 12, wherein the cutter cannula further includes a spindle and a driven gear for translating the cutter cannula between an extended position and a fully retracted position.

14. The biopsy device of claim 13, wherein the spindle engages the driven gear when the cutter cannula is in the extended position, and the spindle engages the spindle gear of the integration module when the cutter cannula is in the fully retracted position.

15. The biopsy device of claim 14, wherein, when the cutter cannula is in the fully retracted position and the marker delivery cartridge is in the delivery position, rotation of the bevel drum causes the pushrod to unwind, such that the pushrod extends through the chamber of the marker delivery cartridge and forces at least one of the plurality of markers through the cutter cannula and out of the sample notch cannula.

16. The biopsy device of claim 14, wherein the biopsy probe assembly further includes an inlet and the transmission cover further includes an adapter, the adapter being configured to engage the inlet and couple the marker delivery assembly to the biopsy probe assembly.

17. The marker delivery assembly of claim 12, wherein the marker delivery cartridge further comprises a ramp positioned in the chamber.

18. The marker delivery assembly of claim 17, wherein, when the cutter cannula is in the fully retracted position and the marker delivery cartridge is in the delivery position, rotation of the bevel drum causes the pushrod to unwind, such that the pushrod extends through the chamber of the marker delivery cartridge and forces at least one of the plurality of markers and the ramp through the cutter cannula.

19. A method of using a biopsy device, the method comprising:
releasably coupling a marker delivery assembly to the biopsy device, the marker delivery assembly including a bevel drum coupled to a pushrod, and a marker delivery cartridge rotatable between an initial position and a delivery position;
rotating the marker delivery cartridge from the initial position to the delivery position;
rotating the bevel drum, such that the rotation of the bevel drum extends the pushrod into and through the marker delivery cartridge;
contacting, with the pushrod, at least one of a plurality of markers housed within the marker delivery cartridge;
pushing, with the pushrod, the least one of the plurality of markers housed within the marker delivery cartridge out of the marker delivery cartridge and into the biopsy device; and
deploying, using the pushrod, the at least one of the plurality of markers from the biopsy device.

20. The method of claim 19, further comprising repositioning the biopsy device at a second target site and deploying another of the at least one of the plurality of markers.
